SEQUENCE LISTING (57 sequences)

SEQ ID NO: 1 - protein, 8 aa, Artificial Sequence
  Synthetic
NQVDQVGY

SEQ ID NO: 2 - protein, 8 aa, Artificial Sequence
  Synthetic
SRPQGPFL

SEQ ID NO: 3 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 4 - protein, 34 aa, Homo sapiens
DACNVNIFDAIAEIGNQLYLFKDGKYWRFSEGRG

SEQ ID NO: 5 - protein, 133 aa, Homo sapiens
IADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSEL

SEQ ID NO: 6 - protein, 11 aa, Homo sapiens
VTYDILQCPED

SEQ ID NO: 7 - protein, 7 aa, Artificial Sequence
  Synthetic
NQVDQVG

SEQ ID NO: 8 - protein, 6 aa, Artificial Sequence
  Synthetic
NQVDQV

SEQ ID NO: 9 - protein, 5 aa, Artificial Sequence
  Synthetic
NQVDQ

SEQ ID NO: 10 - protein, 4 aa, Artificial Sequence
  Synthetic
NQVD

SEQ ID NO: 11 - protein, 7 aa, Artificial Sequence
  Synthetic
QVDQVGY

SEQ ID NO: 12 - protein, 6 aa, Artificial Sequence
  Synthetic
VDQVGY

SEQ ID NO: 13 - protein, 5 aa, Artificial Sequence
  Synthetic
DQVGY

SEQ ID NO: 14 - protein, 4 aa, Artificial Sequence
  Synthetic
QVGY

SEQ ID NO: 15 - protein, 6 aa, Artificial Sequence
  Synthetic
QVDQVG

SEQ ID NO: 16 - protein, 5 aa, Artificial Sequence
  Synthetic
VDQVG

SEQ ID NO: 17 - protein, 4 aa, Artificial Sequence
  Synthetic
DQVG

SEQ ID NO: 18 - protein, 5 aa, Artificial Sequence
  Synthetic
QVDQV

SEQ ID NO: 19 - protein, 4 aa, Artificial Sequence
  Synthetic
VDQV

SEQ ID NO: 20 - protein, 4 aa, Artificial Sequence
  Synthetic
QVDQ

SEQ ID NO: 21 - protein, 7 aa, Artificial Sequence
  Synthetic
SRPQGPF

SEQ ID NO: 22 - protein, 6 aa, Artificial Sequence
  Synthetic
SRPQGP

SEQ ID NO: 23 - protein, 5 aa, Artificial Sequence
  Synthetic
SRPQG

SEQ ID NO: 24 - protein, 4 aa, Artificial Sequence
  Synthetic
SRPQ

SEQ ID NO: 25 - protein, 7 aa, Artificial Sequence
  Synthetic
RPQGPFL

SEQ ID NO: 26 - protein, 6 aa, Artificial Sequence
  Synthetic
PQGPFL

SEQ ID NO: 27 - protein, 5 aa, Artificial Sequence
  Synthetic
QGPFL

SEQ ID NO: 28 - protein, 4 aa, Artificial Sequence
  Synthetic
GPFL

SEQ ID NO: 29 - protein, 6 aa, Artificial Sequence
  Synthetic
RPQGPF

SEQ ID NO: 30 - protein, 5 aa, Artificial Sequence
  Synthetic
PQGPF

SEQ ID NO: 31 - protein, 4 aa, Artificial Sequence
  Synthetic
QGPF

SEQ ID NO: 32 - protein, 5 aa, Artificial Sequence
  Synthetic
RPQGP

SEQ ID NO: 33 - protein, 4 aa, Artificial Sequence
  Synthetic
PQGP

SEQ ID NO: 34 - protein, 4 aa, Artificial Sequence
  Synthetic
RPQG

SEQ ID NO: 35 - protein, 8 aa, Artificial Sequence
  Synthetic
GLSQPRFP

SEQ ID NO: 36 - DNA, 35 bp, Artificial Sequence
  Synthetic
cggaattccgccaacatgagcctctggcagcccct

SEQ ID NO: 37 - DNA, 36 bp, Artificial Sequence
  Synthetic
ggaagatctctagtcctcagggcactgcaggatgtc

SEQ ID NO: 38 - DNA, 67 bp, Artificial Sequence
  Synthetic
ggggtcccagacctgggcagatacccctacgacgtgcccgactcgcctccaaacctttgagggcgac

SEQ ID NO: 39 - DNA, 69 bp, Artificial Sequence
  Synthetic
gtcgccctcaaaggtttggaaggcgtagtcgggcacgtcgtaggggtatctgcccaggtctgggacccc

SEQ ID NO: 40 - DNA, 45 bp, Artificial Sequence
  Synthetic
tacaatgtcctgtttgcagatctcgtccaccggactcaaaggcac

SEQ ID NO: 41 - DNA, 30 bp, Artificial Sequence
  Synthetic
gagatctgcaaacaggacattgtatttgat

SEQ ID NO: 42 - DNA, 33 bp, Artificial Sequence
  Synthetic
cccaagcttctagcagcctagccagtcggattt

SEQ ID NO: 43 - DNA, 33 bp, Artificial Sequence
  Synthetic
gggagccggccggggcccctgctgatcgccgac

SEQ ID NO: 44 - DNA, 33 bp, Artificial Sequence
  Synthetic
gtcggcgatcagcaggggccccggccggctccc

SEQ ID NO: 45 - DNA, 57 bp, Artificial Sequence
  Synthetic
gtgtacacaggcgcgaccttggagcgagggtaccccaagccactggacaagctgggc

SEQ ID NO: 46 - DNA, 57 bp, Artificial Sequence
  Synthetic
cacatgtgtccgcgctggaacctcgctcccatggggttcggtgacctgttcgacccg

SEQ ID NO: 47 - DNA, 62 bp, Artificial Sequence
  Synthetic
gttcgacgtgaaggcgaagaaaatggatcctggcttccccaagctcgtggaccggatgttcc

SEQ ID NO: 48 - DNA, 62 bp, Artificial Sequence
  Synthetic
ggaacatccggtccacgagcttggggaagccaggatccattttcttcgccttcacgtcgaac

SEQ ID NO: 49 - DNA, 56 bp, Artificial Sequence
  Synthetic
gttcccggagtgagttgaagagcgtgaagtttggaagcgtgacctatgacatcctg

SEQ ID NO: 50 - DNA, 56 bp, Artificial Sequence
  Synthetic
caggatgtcataggtcacgcttccaaacttcacgctcttcaactcactccgggaac

SEQ ID NO: 51 - protein, 8 aa, Artificial Sequence
  Synthetic
VQYDNGQV

SEQ ID NO: 52 - protein, 7 aa, Homo sapiens
KPMGPLL

SEQ ID NO: 53 - protein, 9 aa, Homo sapiens
TLERGYPKP

SEQ ID NO: 54 - protein, 10 aa, Homo sapiens
KKMDPGFPKL

SEQ ID NO: 55 - protein, 7 aa, Homo sapiens
KSVKFGS

SEQ ID NO: 56 - protein, 194 aa, Homo sapiens
DACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 57 - protein, 33 aa, Homo sapiens
IADKWPALPRKLDSVFEERLSKKLFFFSGRQVW

(19) United States
(12) Patent Application Publication
(10) Pub. No.: US 2013/0012451 A1
(43) Pub. Date: Jan. 10, 2013

(54) COMPOSITIONS AND METHODS FOR INHIBITING MMP-9-MEDIATED CELL MIGRATION

(75) Inventors: Jian Cao, S. Setauket, NY (US); Antoine Dufour, Vancouver (CA)

(73) Assignee: The Research Foundation of State University of New York

(21) Appl. No.: 13/518,289

(22) PCT Filed: Dec. 20, 2010
(86) PCT No.: PCT/US10/61339

§ 371 Date: Sep. 10, 2012

Related U.S. Application Data
(60) Provisional application No. 61/288,540, filed on Dec. 21, 2009.

(51) Int. Cl. A61K 38/10; C07K 7/06; C12Q 1/02; A61P 35/04; C12N 5/071; C07K 7/08; A61K 38/08

(52) U.S. Cl. 514/19.8; 530/326; 530/328; 530/327; 514/21.4; 514/21.5; 514/21.6; 435/375; 435/29

(57) ABSTRACT

The invention provides peptides, portions and derivatives thereof, that are useful for reducing cell migration, and for reducing symptoms of pathological diseases that are associated with undersirable cell migration, and in particular MMP-9-induced cell migration.

This invention was made with government support under grant 5RO1CA11355301A1, awarded by the National Institutes of Health (NIH). The government has certain rights in the invention.

### FIELD OF INVENTION

The invention provides peptides, portions and derivatives thereof, that are useful for reducing cell migration, and for reducing symptoms of pathological diseases that are associated with undersirable cell migration, and in particular MMP-9-induced cell migration.

### BACKGROUND

Several diseases involve undesired cell migration, including cancer, systemic lupus erythematosus (SLE), Sjogren's syndrome (SS), systemic sclerosis (SS), polymyositis, rheumatoid arthritis (RA), multiple sclerosis (MS), atherosclerosis, cerebral ischemia, abdominal aortic aneurysm (AAA), myocardial infarction (MI), cerebral amyloid angiopathy (CAA), angiogenesis, inflammation, and eczema. These diseases are the cause of loss of life and/or loss of the quality of life. While some therapeutic approaches have been successful, these diseases have not been completely eradicated. For example, cancer metastasis is responsible for 90% of treatment failure among cancer patients. Thus, there remains a need for development of novel treatment strategies to reduce diseases involving undesired cell migration, to improve the quality of life, and to prolong the survival, of patients suffering from these diseases.

### SUMMARY OF THE INVENTION

The invention provides a composition comprising a polypeptide that 1) comprises a sequence selected from the group consisting of at least a portion of NQVDQVGY (SEQ ID NO:1) and at least a portion of SRPQGPFL (SEQ ID NO:2), and 2) lacks at least a portion of MMP-9 hemopexin domain sequence, wherein the portion of MMP-9 hemopexin domain is selected from the group consisting of a) at least a portion of DACNVNIFDAIAEIGNQLYLFKDGKYWRFSEGRG (SEQ ID NO:4), b) at least a portion of IADKWPALPRKLDSVFEERLSKKLFFFSGRQVWVYTGASVLGPRRLDKLGLGADVA QVTGALRSGRGKMLLFSGRRLWRFDVKAQMVDPRSASEVDRMFPGVPLDTHDVFQ YREKAYFCQDRFYWRVSSRSEL (SEQ ID NO:5), and c) at least a portion of VTYDILQCPED (SEQ ID NO:6). While not limiting the portion to any particular sequence, in one embodiment, the portion of NQVDQVGY (SEQ ID NO:1) is selected from NQVDQVG (SEQ ID NO: 7), NQVDQV (SEQ ID NO: 8), NQVDQ (SEQ ID NO: 9), NQVD (SEQ ID NO: 10), QVDQVGY (SEQ ID NO: 11), VDQVGY (SEQ ID NO: 12), DQVGY (SEQ ID NO: 13), QVGY (SEQ ID NO: 14), QVDQVG (SEQ ID NO: 15), VDQVG (SEQ ID NO: 16) DQVG (SEQ ID NO: 17), QVDQV (SEQ ID NO: 18), VDQV (SEQ ID NO: 19), and QVDQ (SEQ ID NO: 20). In another embodiment, the portion of SRPQGPFL (SEQ ID NO:2) is selected from the group consisting of SRPQGPF (SEQ ID NO: 21), SRPQGP (SEQ ID NO: 22), SRPQG (SEQ ID NO: 23), SRPQ (SEQ ID NO: 24), RPQGPFL (SEQ ID NO: 25), PQGPFL (SEQ ID NO: 26), QGPFL (SEQ ID NO: 27), GPFL (SEQ ID NO: 28), RPQGPF (SEQ ID NO: 29), PQGPF (SEQ ID NO: 30), QGPF (SEQ ID NO: 31), RPQGP (SEQ ID NO: 32), PQGP (SEQ ID NO: 33), and RPQG (SEQ ID NO: 34).

The invention also provides a composition comprising a polypeptide that consists of a sequence selected from the group consisting of at least a portion of NQVDQVGY (SEQ ID NO:1) and at least a portion of SRPQGPFL (SEQ ID NO:2). In one embodiment, the polypeptide has been modified to resist proteolysis. In a particular embodiment, the polypeptide has been terminally modified.

Also provided by the invention is a method for reducing one or more symptoms of disease in a subject, comprising a) providing i) a mammalian subject in need of reducing one or more symptoms of disease, and ii) any one or more of the compositions described herein (as exemplified by a first composition comprising a polypeptide that 1) comprises a sequence selected from the group consisting of at least a portion of NQVDQVGY (SEQ ID NO:1) and at least a portion of SRPQGPFL (SEQ ID NO:2), and 2) lacks at least a portion of MMP-9 hemopexin domain sequence, wherein the portion of MMP-9 hemopexin domain is selected from the group consisting of a) at least a portion of DACNVNIFDAIAEIGNQLYLFKDGKYWRFSEGRG (SEQ ID NO:4), b) at least a portion of IADKWPALPRKLDSVFEERLSKKLFFFSGRQVWVYTGASVLGPRRLDKLGLGADVA QVTGALRSGRGKMLLF SGRRLWRFDVKAQMVDPRSASEVDRMFPGVPLDTHDVFQ YREKAYFCQDRFYWRVSSRSEL (SEQ ID NO:5), and c) at least a portion of VTYDILQCPED (SEQ ID NO:6), and as exemplified by a second composition comprising a polypeptide that consists of a sequence selected from the group consisting of at least a portion of NQVDQVGY (SEQ ID NO:1) and at least a portion of SRPQGPFL (SEQ ID NO:2)), and b) administering to the subject a therapeutic amount of the composition to produce a treated subject, wherein the administering is under conditions for reducing one or more symptoms of the disease. In one embodiment, the method further comprises c) detecting a reduction in one or more symptoms of the disease in the treated subject. In a particular embodiment, one or more symptoms of the disease comprise increased cell migration in the presence of MMP-9 compared to in the absence of MMP-9. In yet another embodiment, the therapeutic amount of the composition specifically reduces the cell migration. In an alternative embodiment, the composition comprises an amount of at least a portion of NQVDQVGY (SEQ ID NO:1) that reduces homodimerization of MMP-9. In another embodiment, the composition comprises an amount of at least a portion of SRPQGPFL (SEQ ID NO:2) that reduces heterodimerization of MMP-9 and CD44. In a particular embodiment, the cell is a cancer cell, as exemplified by, but not limited to, a metastatic cancer cell.

The invention additionally provides a method for reducing cell migration, comprising a) providing i) a cell expressing MMP-9, and ii) any one or more of the compositions described herein (as exemplified by a first composition comprising a polypeptide that 1) comprises a sequence selected from the group consisting of at least a portion of NQVDQVGY (SEQ ID NO:1) and at least a portion of SRPQGPFL (SEQ ID NO:2), and 2) lacks at least a portion of MMP-9 hemopexin domain sequence, wherein the portion of MMP-9 hemopexin domain is selected from the group consisting of a) at least a portion of DACNVNIFDAIAEIGNQLYLFKDGKYWRFSEGRG (SEQ ID NO:4), b) at least a portion of IADKWPALPRKLDSVFEERLSKKLFFFSGRQVWVYTGASVLGPRRLDKLGLGADVA QVTGALRSGRGKMLLFSGRRLWRFDVKAQMVDPRSASEVDRMFPGVPLDTHDVFQ YREKAYFCQDRFYWRVSSRSEL (SEQ ID NO:5), and c) at least a portion of VTYDILQCPED (SEQ ID NO:6), and as exemplified by a second composition comprising a polypeptide that consists of a sequence selected from the group consisting of at least a portion of NQVDQVGY (SEQ ID NO:1) and at least a portion of SRPQGPFL (SEQ ID NO:2)), and b) administering the composition to the cell under conditions for reducing migration of the cell. In one embodiment, the method further comprises c) detecting reduced migration of the cell. In a particular embodiment, the migration of the cell is increased in the presence of MMP-9 compared to in the absence of MMP-9, and the composition specifically reduces the cell migration. In a particular embodiment, the composition comprises an amount of at least a portion of NQVDQVGY (SEQ ID NO:1) that reduces homodimerization of MMP-9. In another embodiment, the composition comprises an amount of at least a portion of SRPQGPFL (SEQ ID NO:2) that reduces heterodimerization of MMP-9 and CD44.

Also provided herein is a method for treating a cancer at risk of metastases in a subject, comprising a) providing i) a mammalian subject having cancer at risk of metastases, and ii) any one or more of the compositions described herein (as exemplified by a first composition comprising a polypeptide that 1) comprises a sequence selected from the group consisting of at least a portion of NQVDQVGY (SEQ ID NO:1) and at least a portion of SRPQGPFL (SEQ ID NO:2), and 2) lacks at least a portion of MMP-9 hemopexin domain sequence, wherein the portion of MMP-9 hemopexin domain is selected from the group consisting of a) at least a portion of DACNVNIFDAIAEIGNQLYLFKDGKYWRFSEGRG (SEQ ID NO:4), b) at least a portion of IADKWPALPRKLD SVFEERLSKKLFFFSGRQVWVYTGASVLGPRRLDKLGLGADVA QVTGALRSGRGKMLLFSGRRLWRFDVKAQMVDPRSASEVDRMFPGVPLDTHDVFQ YREKAYFCQDRFYWRVSSRSEL (SEQ ID NO:5), and c) at least a portion of VTYDILQCPED (SEQ ID NO:6), and as exemplified by a second composition comprising a polypeptide that consists of a sequence selected from the group consisting of at least a portion of NQVDQVGY (SEQ ID NO:1) and at least a portion of SRPQGPFL (SEQ ID NO:2)), and b) administering to the subject a therapeutic amount of the composition to produce a treated subject, wherein the administering is under conditions for reducing the risk of metastases.

### BRIEF DESCRIPTION OF THE DRAWINGS

FIG. 1. MMP-9 homodimerizes through its PEX domain. (A) Ribbon diagram of MMP-9 PEX domain (PDB: 1ITV). Dimerization of recombinant MMP-9 PEX is through the fourth blade. (B) Schematic diagram of wild type MMP-9, MMP-9/HA, MMP-9/Myc and MMP9/PEX_{MMP2}/Myc. (C) MMP-9 forms a homodimer in COS-1 cells transfected with MMP-9/Myc and MMP-9/HA cDNAs followed by a co-immuneprecipitation assay (upper panel) and reciprocal co-immuneprecipitation (lower panel). 20 μg of total cell lysates were used as loading control by anti-α/β tubulin antibody. (D) Replacement of MMP-9 PEX domain by the corresponding region of MMP-2 failed to dimerize with wild type MMP-9 examined by a co-immuneprecipitation assay.

FIG. 2. MMP-9 homodimer is required for MMP-9-enhanced cell migration. COS-1 cells transfected with wild type and mutant MMP-9 cDNAs were examined by a transwell chamber migration assay (A) and phagokinetic assay (B). Migratory ability of cells was quantitatively determined (C). (P-values reflect comparison with MMP-9/Myc transfected cells: *P<0.05).

FIG. 3. TIMP-1 interferes with MMP-9 homodimerization. (A) TIMP-1, but not TIMP-2 interferes with MMP-9 dimerization in transfected COS-1 cells examined by a co-immunoprecipitation assay for conditioned medium and cell lysates (upper and middle panels), and immunoblotting for α/β tubulin (lower panel, loading control). (B) TIMP-1 co-precipitated with MMP-9 in both the lysate and the conditioned medium of transfected COS-1 cells examined by a co-immunoprecipitation assay. (C) MMP-9 chimera substituting the PEX domain with the corresponding MMP-2 interacts with TIMP-2 in transfected COS-1 cells, but not wild type MMP-9. (D) COS-1 cells transfected with corresponding cDNAs were subjected to a transwell migration assay. Three triplicated repeats were performed for each transfection. *P<0.05.

FIG. 4. Blade IV of the PEX domain of proMMP-9 is required for homodimerization and cell migration. (A) Ribbon diagram of MMP-9 PEX domain (PDB:1ITV). Each outermost β-strand from the four blades was swapped with the corresponding MMP-2 PEX domain sequences (upper panel). Lower panel: Schematic diagram of substitution mutations at the outermost 3-strands of four blade of MMP-9 by corresponding MMP-2 sequences. (B) MMP-9/blade IV mutant (MMP-9/IVS4) does not homodimerize with MMP-9/Myc examined by a co-immunoprecipitation (upper panel) and by immunoblotting using anti-HA antibody (middle panel). The conditioned medium was also examined by gelatin zymography (lower panel). (C) MMP-9/IS4, IVS4 and MMP-9/PEX_{MMP2}, chimera failed to enhance COS-1 cell migration examined by a transwell migration assay. (P-values reflect comparison with MMP-9 transfected cells: *P<0.05). (D) A peptide mimicking MMP-9 IVS4 abrogates MMP-9 dimerization examined by co-immunoprecipitation (upper panel). Total cell lysate (20 μg) was also examined by an immunoblotting assay using anti-HA antibody (lower panel). (E) Dose dependent inhibition of MMP-9-mediated cell migration by IVS4 peptides. No effect was found using scrambled peptides. COS-1 cells transfected with an empty vector or MMP-9 cDNAs were examined by a transwell chamber migration assay for 6 hours in the presence of DMSO control, specific and scramble peptides at different concentration. Each data point was performed in triplicate and the experiments was repeated three times (*P<0.05).

FIG. 5. CD44 serves as a docking molecule for MMP-9 on the cell surface and facilitates MMP-9-mediated cell migration. (A) CD44 forms a complex with MMP-9 in co-transfected COS-1 cells examined by co-immunoprecipitation using anti-CD44 antibody for IP and anti-MMP-9 antibody for IB. (B) Expression of CD44 mRNA in COS-1 cells and CD44-silenced COS-1 cells. Total RNAs were extracted followed by a real time RT PCR analysis. The relative quantitative value CD44 expression was normalized to housekeeping genes HPRT1 and GAPDH (?). Each bar represents the mean±S.E. (C) MMP-9 enhancement of cell migration is dependent on CD44. CD44 silenced COS-1 cells were transfected with MMP-9 or vector control followed by transwell migration assay (upper panel) and immunoblotting assays for MMP-9, CD44 or α/β tubulin (lower panel). (D) Peptides mimicking the outermost β-strand of the blade I interferes with MMP-9 heterodimer formation (upper panel). 20 ng of total cell lysates were examined by immunoblotting using anti-MMP-9 antibody (lower panel). (E) Dose-dependent inhibition of MMP-9-mediated cell migration by IS4 peptides. COS-1 cells transfected with an empty vector or MMP-9 cDNAs were incubated with 1% DMSO, IS4 peptide (SRPQGPFL) (SEQ ID NO: 2) or IS4 scrambled peptide (GLSQPRFP) (SEQ ID NO: 35) for 6 h in a transwell chamber migration assay. Each data point was performed in triplicate and the experiment was repeated three times (*P<0.05).

FIG. 6. CD44 interacts with EGFR (?) to regulate MMP-9 enhanced cell migration. (A) Dose dependent inhibition of MMP-9-mediated cell migration by EGFR inhibitor (AG1478). COS-1 cells transfected with vector or MMP-9 were treated with different concentrations of AG1478 for 30 min before being subjected to a transwell migration assay. (P-values reflect comparison with MMP-9 transfected cells: *P<0.05). (B) Activation of EGFR (?) downstream effectors in COS-1 cells transfected with MMP-9 cDNAs, but not in CD44-silenced COS-1 cells. At 48 h after transfection, cell lysates were prepared and subjected to western blot analysis using antibodies against pERK1/2, ERK1/2, pAKT, AKT, pFAK, FAK, pEGFR, EGFR (?) and α/β-tubulin antibodies.

FIG. 7. Insertion of HA and Myc tags did not affect MMP-9 secretion. (A) Wild type, HA- and Myc-tagged MMP-9 from cDNA transfected COS-1 cells degrade gelatin examined by a gelatin zymography. (B) Expression of MMP-9 and HA- or Myc-tagged MMP-9 in COS-1 cells examined by immunoblotting assays. (C) Antibodies to HA tag or Myc tag efficiently precipitated HA-tagged and Myc-tagged MMP-9 chimera, respectively. COS-1 cells were transfected with cDNAs as indicated. The conditioned media and total cell lysates were immunoprecipitated with anti-HA antibody (upper panel) and anti-Myc antibody (lower panel). (D) Mutant MMP-9 by swapping the PEX domain with that of MMP-2 (MMP9/PEX_{MMP2} expresses comparable level of proteins examined by an immunoblotting assay (upper panel) and digests gelatin examined by gelatin zymography (lower panel). (E) TIMP-1 co-precipitated with MMP-9 in both the lysate and the conditioned medium of transfected COS-1 cells detected by a reciprocal co-immunoprecipitation assay.

FIG. 8. Silencing of CD44 in COS-1 cells using a shRNA approach. (A) Expression of CD44 in COS-1 cells stably transfected with shRNA luciferase (a-c) or CD44 shRNA (d-f) was analyzed by immunofluorescence staining using anti-CD44 antibodies. Nucleus was counterstained with DAPI (blue). (B) Expression of CD44 in COS-1 cells stably transfected with shRNA luciferase control or CD44 shRNA was analyzed by flow cytometry analysis using anti-CD44 antibody.

FIG. 9. CD44 interacts with EGFR (?) to regulate MMP-9-enhanced cell migration. (A) Densitometric analyses of the levels of phosphorylation of pERK, pAKT, pFAK and pEGFR compared to corresponding pan antibodies. (B) Increase of phosphorylated cofilin and paxillin in MMP-9 transfected COS-1 cells. After antibody screening using a Kinexus Antibody Microarray, immunoblotting was employed to validate the antibody array data performed by the Kinexus. (C) Densitometric analyses of the phosphorylation levels of pCofilin 1 and pPaxillin in MMP-9 transfected COS-1 cells compared to vector. The phosphorylation of Cofilin 1 is increased by 30% and 54% for Paxillin in MMP-9 transfected cells compared to vector control.

FIG. 10. An exemplary full-length amino acid sequence of MMP-9 (SEQ ID NO:3), with the IVS4 peptide sequence N₅₈₉QVDQVGY₆₉₆ (SEQ ID NO:1) (?) and IS4 peptide sequence S₅₄₈RPQGPFL₅₅₅ (SEQ ID NO:2) (?) in bold character within the hemopexin domain.

FIG. 11 Inhibition of HT1080 cell migration by synthetic peptides interfering with MMP-9 dimerizations.

### DEFINITIONS

To facilitate understanding of the invention, a number of terms are defined below.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed using a recombinant DNA molecule.

The terms “endogenous” and “wild type” when in reference to a sequence refer to a sequence which is naturally found in the cell or virus into which it is introduced so long as it does not contain some modification relative to the naturally-occurring sequence. The term “heterologous” refers to a sequence that is not endogenous to the cell or virus into which it is introduced.

The terms “mutation” and “modification” refer to a deletion, insertion, or substitution.

A “deletion” is defined as a change in a nucleic acid sequence or amino acid sequence in which one or more nucleotides or amino acids, respectively, is absent.

An “insertion” or “addition” is that change in a nucleic acid sequence or amino acid sequence that has resulted in the addition of one or more nucleotides or amino acids, respectively.

A “substitution” in a nucleic acid sequence or an amino acid sequence results from the replacement of one or more nucleotides or amino acids, respectively, by a molecule that is a different molecule from the replaced one or more nucleotides or amino acids. For example, a nucleic acid may be replaced by a different nucleic acid as exemplified by replacement of a thymine by a cytosine, adenine, guanine, or uridine. Alternatively, a nucleic acid may be replaced by a modified nucleic acid as exemplified by replacement of a thymine by thymine glycol. Substitution of an amino acid may be conservative or non-conservative. “Conservative substitution” of an amino acid refers to the replacement of that amino acid with another amino acid which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains that may be conservatively substituted one for the other include phenylalanine, tyrosine, and tryptophan. Cysteine and methionine are sulphur-containing amino acids which may be conservatively substituted one for the other. Also, asparagine may be conservatively substituted for glutamine, and vice versa, since both amino acids are amides of dicarboxylic amino acids. In addition, aspartic acid (aspartate) may be conservatively substituted for glutamic acid (glutamate) as both are acidic, charged (hydrophilic) amino acids. Also, lysine, arginine, and histidine may be conservatively substituted one for the other since each is a basic, charged (hydrophilic) amino acid. “Non-conservative substitution” is a substitution other than a conservative substitution. Guidance in determining which and how many amino acid residues may be substituted, inserted or deleted without abolishing biological and/or immunological activity may be found using computer programs well known in the art, for example, DNAStar™ software.

A “variant” or “homolog” of an amino acid sequence of interest refers to an amino acid sequence that differs by insertion, deletion, and/or conservative substitution of one or more amino acids from the amino acid sequence of interest. In one embodiment, the variant sequence has at least 95% identity, including at least 90% identity, at least 85% identity, at least 80% identity, at least 75% identity, at least 70% identity, and/or at least 65% identity with the amino acid sequence of interest. Guidance in determining which and how many amino acid residues may be substituted, inserted or deleted without abolishing biological and/or immunological activity may be found using computer programs well known in the art, for example, DNAStar™ software.

A “variant” or “homolog” of a nucleotide sequence of interest refers to a nucleotide sequence that differs by insertion, deletion, and/or substitution of one or more nucleotides from the nucleotide sequence of interest. In one embodiment, the variant sequence has at least 95% identity, including at least 90% identity, at least 85% identity, at least 80% identity, at least 75% identity, at least 70% identity, and/or at least 65% identity with the nucleotide sequence of interest.

The term “expression vector” as used herein refers to a recombinant DNA molecule containing a desired coding sequence and appropriate nucleic acid sequences necessary for the expression (i.e., transcription and/or translation) of the operably linked coding sequence in a particular host organism. Expression vectors are exemplified by, but not limited to, plasmid, phagemid, shuttle vector, cosmid, virus, chromosome, mitochondrial DNA, plastid DNA, and nucleic acid fragment. Nucleic acid sequences used for expression in prokaryotes include a promoter, optionally an operator sequence, a ribosome binding site and possibly other sequences. Eukaryotic cells are known to utilize promoters, enhancers, and termination and polyadenylation signals.

The terms “purified,” “isolated,” and grammatical equivalents thereof as used herein, refer to the reduction in the amount of at least one undesirable component (such as cell type, protein, and/or nucleic acid sequence) from a sample, including a reduction by any numerical percentage of from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100%. Thus purification results in an “enrichment,” i.e., an increase in the amount of a desirable cell type, protein and/or nucleic acid sequence in the sample.

The terms “operable combination” and “operably linked” when in reference to the relationship between nucleic acid sequences and/or amino acid sequences refers to linking the sequences such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest resulting in an mRNA that directs the synthesis of a polypeptide encoded by the nucleotide sequence of interest.

As used herein, the terms “treat”, “treating”, “treatment” and grammatical equivalents refers to combating a disease or disorder, as for example in the management and care of a patient. In one embodiment, treating a disease (e.g., cancer, metastasis, etc.) includes reducing one or more symptoms of the disease.

As used herein, the terms “diagnose”, “diagnosis” or “diagnosing” refers to the recognition of a disease by its signs and symptoms (e.g., resistance to conventional therapies), or genetic analysis, pathological analysis, histological analysis, and the like.

As used herein, the term “diagnostic” refers to a compound that assists in the identification and characterization of a health or disease state. The diagnostic can be used in standard assays as is known in the art.

As used herein, the terms “cancer cell” and “tumor cell” refer to a cell undergoing early, intermediate or advanced stages of multi-step neoplastic progression as previously described (Pitot et al., Fundamentals of Oncology, 15-28 (1978)), herein incorporated by reference. The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. A cell in the early stages of malignant progression is referred to as a “hyperplastic cell” and is characterized by dividing without control and/or at a greater rate than a normal cell of the same cell type in the same tissue. Proliferation may be slow or rapid but continues unabated. A cell in the intermediate stages of neoplastic progression is referred to as a “dysplastic cell”. A dysplastic cell resembles an immature epithelial cell, is generally spatially disorganized within the tissue and loses its specialized structures and functions. During the intermediate stages of neoplastic progressions an increasing percentage of the epithelium becomes composed of dysplastic cells. “Hyperplastic” and “dysplastic” cells are referred to as “pre-neoplastic” cells. In the advanced stages of neoplastic progression a dysplastic cell become a “neoplastic” cell. Neoplastic cells are typically invasive i.e., they either invade adjacent tissues, or are shed from the primary site and circulate through the blood and lymph to other locations in the body where they initiate secondary cancers.

The term “cancer” or “neoplasia” refers to a plurality of cancer cells.

A “cancer at risk for metastases” refers to a cancer that may differentiate into a metastatic cancer. Such risk may be based on family history, genetic factors, type of cancer, environmental factors, etc.

“Carcinoma” refers to a malignant new growth made up of epithelial cells tending to infiltrate the surrounding tissues and give rise to metastases.

“Metastatic” cancer cell refers to a cancer cell that is translocated from a primary cancer site (i.e., a location where the cancer cell initially formed from a normal, hyperplastic or dysplastic cell) to a site other than the primary site, where the translocated cancer cell lodges and proliferates.

The terms “therapeutic amount,” “pharmaceutically effective amount,” “therapeutically effective amount,” and “biologically effective amount,” are used interchangeably herein to refer to an amount that is sufficient to achieve a desired result, whether quantitative or qualitative. In particular, a pharmaceutically effective amount is that amount that results in the reduction, delay, and/or elimination of undesirable effects (such as pathological, clinical, biochemical and the like) in the subject that are associated with disease. For example, a “therapeutic amount that reduces cancer metastasis” is an amount that that reduces, delays, and/or eliminates one or more symptoms of cancer metastasis. Also, a “therapeutic amount that reduces one or more symptoms of cancer” is an amount that reduces, delays, and/or eliminates one or more symptoms of cancer. The actual amount encompassed by the term “therapeutic amount” will depend on the route of administration, the type of subject being treated, and the physical characteristics of the specific subject under consideration. These factors and their relationship to determining this amount are well known to skilled practitioners in the medical, veterinary, and other related arts and are further discussed herein.

The terms “reduce,” “inhibit,” “diminish,” “suppress,” “decrease,” and grammatical equivalents (including “lower,” “smaller,” etc.) when in reference to the level of any molecule (e.g., amino acid sequence, and nucleic acid sequence such as those encoding any of the polypeptides described herein), cell, and/or phenomenon (e.g., cell migration, disease symptom, heterodimerization, homodimerization, binding to a molecule, affinity of binding, expression of a nucleic acid sequence, transcription of a nucleic acid sequence, enzyme activity, etc.) in a first sample (or in a first subject) relative to a second sample (or relative to a second subject), mean that the quantity of molecule, cell and/or phenomenon in the first sample (or in the first subject) is lower than in the second sample (or in the second subject) by any amount that is statistically significant using any art-accepted statistical method of analysis. In one embodiment, the quantity of molecule, cell and/or phenomenon in the first sample (or in the first subject) is at least 10% lower than, at least 25% lower than, at least 50% lower than, at least 75% lower than, and/or at least 90% lower than the quantity of the same molecule, cell and/or phenomenon in the second sample (or in the second subject). In another embodiment, the quantity of molecule, cell, and/or phenomenon in the first sample (or in the first subject) is lower by any numerical percentage from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100% lower than the quantity of the same molecule, cell and/or phenomenon in the second sample (or in the second subject). In one embodiment, the first subject is exemplified by, but not limited to, a subject to whom the invention's compositions have been administered. In a further embodiment, the second subject is exemplified by, but not limited to, a subject to whom the invention's compositions have not been administered. In an alternative embodiment, the second subject is exemplified by, but not limited to, a subject to whom the invention's compositions have been administered at a different dosage and/or for a different duration and/or via a different route of administration compared to the first subject. In one embodiment, the first and second subjects may be the same individual, such as where the effect of different regimens (e.g., of dosages, duration, route of administration, etc.) of the invention's compositions is sought to be determined in one individual. In another embodiment, the first and second subjects may be different individuals, such as when comparing the effect of the invention's compositions on one individual participating in a clinical trial and another individual in a hospital.

The terms “increase,” “elevate,” “raise,” and grammatical equivalents (including “higher,” “greater,” etc.) when in reference to the level of any molecule (e.g., amino acid sequence, and nucleic acid sequence such as those encoding any of the polypeptides described herein), cell, and/or phenomenon (e.g., cell migration, disease symptom, heterodimerization, homodimerization, binding to a molecule, affinity of binding, expression of a nucleic acid sequence, transcription of a nucleic acid sequence, enzyme activity, etc.) in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis. In one embodiment, the quantity of the molecule, cell and/or phenomenon in the first sample (or in the first subject) is at least 10% greater than, at least 25% greater than, at least 50% greater than, at least 75% greater than, and/or at least 90% greater than the quantity of the same molecule, cell and/or phenomenon in the second sample (or in the second subject). This includes, without limitation, a quantity of molecule, cell, and/or phenomenon in the first sample (or in the first subject) that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule, cell and/or phenomenon in the second sample (or in the second subject). In one embodiment, the first subject is exemplified by, but not limited to, a subject to whom the invention's compositions have been administered. In a further embodiment, the second subject is exemplified by, but not limited to, a subject to whom the invention's compositions have not been administered. In an alternative embodiment, the second subject is exemplified by, but not limited to, a subject to whom the invention's compositions have been administered at a different dosage and/or for a different duration and/or via a different route of administration compared to the first subject. In one embodiment, the first and second subjects may be the same individual, such as where the effect of different regimens (e.g., of dosages, duration, route of administration, etc.) of the invention's compositions is sought to be determined in one individual. In another embodiment, the first and second subjects may be different individuals, such as when comparing the effect of the invention's compositions on one individual participating in a clinical trial and another individual in a hospital.

Reference herein to any numerical range expressly includes each numerical value (including fractional numbers and whole numbers) encompassed by that range. To illustrate, and without limitation, reference herein to a range of “at least 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes each whole number of 5, 6, 7, 8, 9, and 10, and each fractional number such as 5.1, 5.2, 5.3, 5.4, 5.5, 5.6, 5.7, 5.8, 5.9, etc.

“Matrix metalloproteinases” are a family of nine or more highly homologous Zn(++)-endopeptidases that collectively cleave most if not all of the constituents of the extracellular matrix, and act on pro-inflammatory cytokines, chemokines and other proteins to regulate varied aspects of inflammation and immunity. Emerging evidence has emphasized the role of matrix metalloproteinases (MMPs) in early aspects of cancer metastasis. Inhibition of proteolytic activity has been a long-term focus of MMP research. A decade ago, the pharmaceutical industry launched ambitious programs to develop MMP inhibitors (MMPI) for the treatment of cancer. Based on the assumption that degradation of collagen by MMPs is considered to be an essential component in progression from in situ carcinoma to invasive/metastatic cancer, the initial anti-MMP drugs for use in cancer were designed as peptide mimics of the collagen amino-acid sequence surrounding the collagenase cleavage site. These MMPIs bind to the catalytic site of MMPs and interfere with their proteolytic activity. Although these MMPIs were successful in interfering with cancer growth and dissemination in animal models, the use of these broad spectrum MMPIs in randomized clinical trials of patients with advanced cancers showed a lack of efficacy. Since the catalytic domain of all MMPs shares highly conserved sequence, lack of specificity of developed MMP enzymatic inhibitors has hindered MMP inhibitor drug discovery. Based on the lessons learned from MMP inhibitor clinical trials, alternative MMP inhibition strategies, based on blocking cell-surface interactions and activation, should form the basis of future therapies for targeting these enzymes in cancer prevention/treatment. A major conceptual advance in development of novel MMPIs to target non catalytic functions of the proteases.

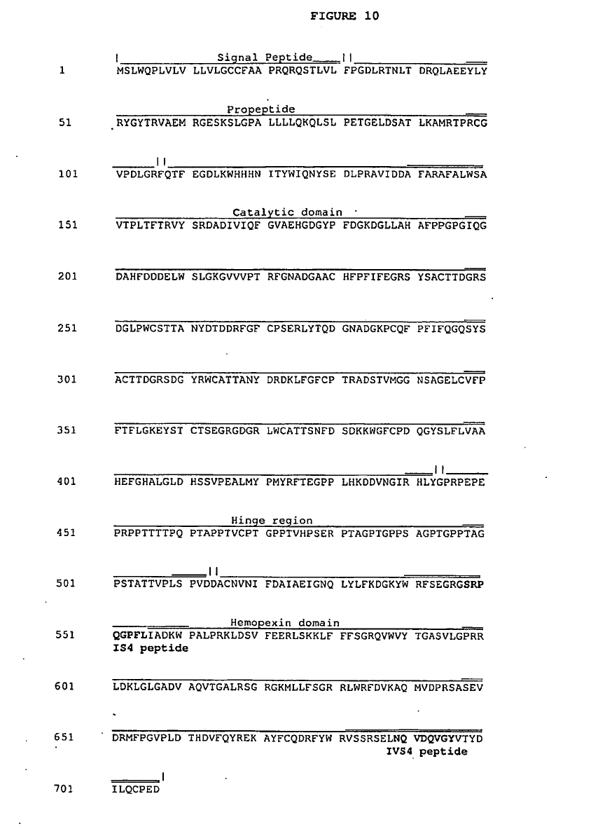

“Matrix Metalloproteinase-9” and “MMP-9” refer to a Matrix Metalloproteinase as exemplified in FIG. 10, that contains a signal peptide, N-terminal propeptide, catalytic domain that contains three fibronectin type II repeats, hinge region, and a C-terminal “hemopexin domain,” also referred to as “PEX domain” and “MMP-9-PEX domain.” In one embodiment, the MMP-9-PEX domain has the sequence shown from D₅₁₄ to D₇₀₇ (SEQ ID NO: 56) of MMP-9 SEQ ID NO:3 (FIG. 10). The invention's IVS4 peptide sequence N₅₈₉QVDQVGY₆₉₆ (SEQ ID NO:1) and IS4 peptide sequence S₅₄₈RPQGPFL₅₅₅ (SEQ ID NO:2) (?) are contained within the MMP-9-PEX domain (FIG. 10).

### BRIEF DESCRIPTION OF THE INVENTION

The invention provides peptides, portions and derivatives thereof, that are useful for reducing cell migration, and for reducing symptoms of pathological diseases that are associated with undersirable cell migration, and in particular MMP-9-induced cell migration.

To develop effective drugs and methods to treat patients with diseases that involve aberrant cell migration (e.g., cancer (including metastatic cancer), systemic lupus erythematosus (SLE), Sjogren's syndrome (SS), systemic sclerosis (SS), polymyositis, rheumatoid arthritis (RA), multiple sclerosis (MS), atherosclerosis, cerebral ischemia, abdominal aortic aneurysm (AAA), myocardial infarction (MI), cerebral amyloid angiopathy (CAA), angiogenesis, inflammation, and eczema), an initial requirement is to identify the weak link in the migrating cell that can be attacked with a specific drug. The inventors have demonstrated that a soluble, tissue degrading enzyme, called MMP-9, can enhance cancer cell migration. Moreover, increased MMP-9 expression was found in various invasive cancers as compared to adjacent normal tissue based on data mining of DNA microarray database. This demonstrates that inhibition of functional MMP-9 represents a useful approach to intervene in diseases involving undesirable cell migration.

The catalytic domain of MMP-9 molecule is required for the enzymatic activity. Since this domain is highly conserved among MMP family members, targeting the catalytic domain has failed in several clinical trials due to lack of selectivity resulting in severe side effects in patients with advanced cancers.

By employing a biochemical approach, the inventors identified another region that is required for MMP-9-enhanced cell migration. Given the fact that cell migration is a critical determinant in some diseases (e.g., cancer invasiveness and metastasis, systemic lupus erythematosus (SLE), Sjogren's syndrome (SS), systemic sclerosis (SS), polymyositis, rheumatoid arthritis (RA), multiple sclerosis (MS), atherosclerosis, cerebral ischemia, abdominal aortic aneurysm (AAA), myocardial infarction (MI), cerebral amyloid angiopathy (CAA), angiogenesis, inflammation, and eczema), targeting MMP-9-enhanced cell migration represents a useful approach to prevent disease. In order to target MMP-9-enhanced cell migration, the inventors have employed molecular techniques to identify a minimal motif within MMP-9 molecule required for cell migration. Based on the identified sequence, the inventors designed and synthesized inhibitory peptides. Using a cell-testing assay to evaluate cell migratory ability, the inventors found that the inhibitory peptides (but not reagents from the scrambled control peptide) efficiently blocked MMP-9-induced cell migration through both homodimerization and heterodimerization. The inventors further demonstrated that the inhibitory peptides are specific for MMP-9-induced cell migration, but not for other MMP-induced cell migration. Therefore, the peptides of the invention can be used to therapeutically intervene in diseases involving undesired cell migration (e.g., cancer metastasis).

### DETAILED DESCRIPTION OF THE INVENTION

The invention provides peptides, portions and derivatives thereof, that are useful for reducing cell migration, and for reducing symptoms of pathological diseases that are associated with undersirable cell migration, and in particular MMP-9-induced cell migration.

Since proteolytic activity is required for cell-cell dissociation/extracellular matrix degradation, and migratory ability is required for cell translocation—the two major determinates for cancer invasion, the inventors' research has been focused on not only proteolytic activity of MMPs, but also cell migratory function of MMPs. The inventors have demonstrated that MMPs play a critical role in cancer cell migration in addition to the proteolytic activity, cell migration is a critical determinant of cancer invasiveness and metastasis. Accordingly, the invention provides compositions and methods for targeting MMP-9-enhanced cell migration as a useful approach to prevent diseases involving undesirable cell migration (e.g., cancer (including cancer metastasis), systemic lupus erythematosus (SLE), Sjogren's syndrome (SS), systemic sclerosis (SS), polymyositis, rheumatoid arthritis (RA), multiple sclerosis (MS), atherosclerosis, cerebral ischemia, abdominal aortic aneurysm (AAA), myocardial infarction (MI), cerebral amyloid angiopathy (CAA), angiogenesis, inflammation, and eczema).

By employing a mutagenesis approach, the inventors recently demonstrated that the hemopexin domain (a non-catalytic domain) of MMP-9 is required for MMP-9-induced cell migration through both homo- and hetero-dimerizations. The inventors further pinpointed minimal motifs within the hemopexin domain required for cell migration. Employing biochemical approaches, the inventors also demonstrated that the defined motif within the blade I of the PEX domain of MMP-9 interacts with the cell surface molecule CD44, which signals for cell migration through cross-talk with a receptor tyrosine kinase, EGFR. Based on this genetic and biochemical data, the inventors designed and synthesized competitive peptides blocking MMP-9 heterodimer and homodimer formations. Two of four specific peptides (IS4 for heterodimer formation: SRPQGPFL (SEQ ID NO:2) and IVS4 for homo-dimer formation: NQVDQVGY (SEQ ID NO:1)) efficiently reduced MMP-9-mediated cell migration in both transfected COS-1 cells and human fibrosarcoma HT1080 cells expressing endogenous MMP-9. Using a Transwell chamber cell migration assay, the inventors demonstrated that the peptides mimicking the essential region of the PEX domain of MMP-9 inhibits MMP-9-induced cell migration in a dose dependent fashion, whereas the control scrambled peptides did not exhibit any inhibitory effect on MMP-9-induced cell migration. Furthermore, the inhibitory peptide specifically inhibits MMP-9-induced cell migration, but not MMP-2 or membrane bound MMP (MT1-MMP)-induced cell migration. This observation indicates that peptides targeting the hemopexin domain of MMPs can achieve the goal of drug selectivity, which is one of the major reasons for the failure of MMPI (MMP catalytic inhibitors) clinical trials.

The invention is further described under A. Exemplary Compositions Of The Invention, B. Exemplary Uses Of The Invention's Compositions, and C. Discussion Of The Exemplary Embodiments In Examples 1-8.

### A. Exemplary Compositions of the Invention

The invention provides compositions comprising a polypeptide having a sequence selected from the group of at least a portion of NQVDQVGY (SEQ ID NO:1) (IVS4) and at least a portion of SRPQGPFL (SEQ ID NO:2) (IS4).

The invention additionally provides a composition comprising a polypeptide that 1) comprises a sequence selected from the group of at least a portion of IVS4 NQVDQVGY (SEQ ID NO:1) and at least a portion of IS4 SRPQGPFL (SEQ ID NO:2), and 2) lacks at least a portion of MMP-9 hemopexin domain sequence. In one embodiment, the portion of MMP-9 hemopexin domain is at least a portion of DACNVNIFDAIAEIGNQLYLFKDGKYWRFSEGRG (SEQ ID NO:4), i.e., from D₅₁₄ to G₅₄₇ (SEQ ID NO: 4) of MMP-9 SEQ ID NO:3. In another embodiment, the portion of MMP-9 hemopexin domain is at least a portion of IADKWPALPRKLDSVFEERLSKKLFFFSGRQVWVYTGASVLGPRRLDKLGLGADVA QVTGALRSGRGKMLLFSGRRLWRFDVKAQMVDPRSASEVDRMFPGVPLDTHDVFQ YREKAYFCQDRFYWRVSSRSEL (SEQ ID NO:5), i.e., from I₅₅₆ to L₅₈₈ (SEQ ID NO: 57) of MMP-9 SEQ ID NO:3. In yet a further embodiment, the portion of MMP-9 hemopexin domain is c) at least a portion of VTYDILQCPED (SEQ ID NO:6), i.e., from V₆₉₇ to D₇₀₇ of MMP-9 SEQ ID NO:3.

The invention's compositions may be used to reduce migration of different cell types in vivo and/or in vitro. These compositions are also useful for reducing one or more symptoms of pathological conditions and/or biochemical processes that involve MMP-9-induced cell migration.

The terms “peptide,” “peptide sequence,” “amino acid sequence,” “polypeptide,” and “polypeptide sequence” are used interchangeably herein to refer to at least two amino acids or amino acid analogs that are covalently linked by a peptide bond or an analog of a peptide bond. The term peptide includes oligomers and polymers of amino acids or amino acid analogs. The term peptide also includes molecules, which are commonly referred to as peptides, which generally contain from about two (2) to about twenty (20) amino acids. The term peptide also includes molecules, which are commonly referred to as polypeptides, which generally contain from about twenty (20) to about fifty amino acids (50). The term peptide also includes molecules, which are commonly referred to as proteins, which generally contain from about fifty (50) to about three thousand (3000) amino acids. The amino acids of the peptide may be L-amino acids or D-amino acids. A peptide, polypeptide or protein may be synthetic, recombinant or naturally occurring. A synthetic peptide is a peptide, which is produced by artificial means in vitro (e.g., was not produced in vivo).

The peptide may be a derivative peptide. The terms “derivative” or “modified” when used in reference to a peptide mean that the peptide contains at least one derivative amino acid. A “derivative” of an amino acid and a “modified” amino acid is a chemically modified amino acid. Derivative amino acids can be “biological” or “non-biological” amino acids. Chemical derivatives of one or more amino acid members may be achieved by reaction with a functional side group. Illustrative derivatized molecules include for example those molecules in which free amino groups have been derivatized to form amine hydrochlorides, p-toluene sulfonyl groups, carboxybenzoxy groups, t-butyloxycarbonyl groups, chloroacetyl groups or formyl groups. Free carboxyl groups may be derivatized to form salts, methyl and ethyl esters or other types of esters and hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine, and ornithine for lysine. Other included modifications are amino terminal acylation (e.g., acetylation or thioglycolic acid amidation), terminal carboxylamidation (e.g., with ammonia or methylamine), and similar terminal modifications. In one embodiment, peptides of the present invention are modified to resist proteolysis. Terminal modifications are useful, as is well known, to reduce susceptibility by (i.e. increases resistance to) proteinase (or protease) digestion and therefore to prolong the half-life of the peptides in solutions, particularly in biological fluids where proteases may be present. Exemplary modified amino acids include, without limitation, 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4-Diaminobutyric acid, Desmosine, 2,2′-Diaminopimelic acid, 2,3-Diaminopropionic acid, N-Ethylgilycine, N-Ethylasparagine, Hydroxylysine, allo-Hydroxylysine, 3-Hydroxyproline, 4-Hydroxyproline, Isodesmosine, allo-Isoleucine, N-Methylglycine, sarcosine, N-Methylisoleucine, N-Methylavaline, Norvaline, Norleucine, and Ornithine Derivatives also include peptides containing one or more additions or deletions as long as the requisite activity is maintained.

The amino acids of the peptides are contemplated to include biological amino acids as well as non-biological amino acids. Accordingly, as used herein, the term “biological amino acid” refers to any one of the known 20 coded amino acids that a cell is capable of introducing into a polypeptide translated from an mRNA. The term “non-biological amino acid,” as used herein, refers to an amino acid that is not a biological amino acid. Non-biological amino acids are useful, for example, because of their stereochemistry or their chemical properties. The non-biological amino acid norleucine, for example, has a side chain similar in shape to that of methionine. However, because it lacks a side chain sulfur atom, norleucine is less susceptible to oxidation than methionine. Other examples of non-biological amino acids include aminobutyric acids, norvaline and allo-isoleucine, that contain hydrophobic side chains with different steric properties as compared to biological amino acids.

Peptides that are useful in the instant invention may be synthesized by several methods, including chemical synthesis and recombinant DNA techniques. Synthetic chemistry techniques, such as solid phase Merrifield synthesis are preferred for reasons of purity, freedom from undesired side products, ease of production, etc. A summary of the techniques available are found in several articles, including Steward et al., Solid Phase Peptide Synthesis, W. H. Freeman, Co., San Francisco (1969); Bodanszky, et al., Peptide Synthesis, John Wiley and Sons, Second Edition (1976); J. Meienhofer, Hormonal Proteins and Peptides, 2:46, Academic Press (1983); Merrifield, Adv. Enzymol. 32:221-96 (1969); Fields, et al., Intl. Peptide Protein Res., 35:161-214 (1990), and U.S. Pat. No. 4,244,946 for solid phase peptide synthesis; and Schroder et al., The Peptides, Vol 1, Academic Press (New York) (1965) for classical solution synthesis. Protecting groups usable in synthesis are described as well in Protective Groups in Organic Chemistry, Plenum Press, New York (1973). Solid phase synthesis methods consist of the sequential addition of one or more amino acid residues or suitably protected amino acid residues to a growing peptide chain. Either the amino or carboxyl group of the first amino acid residue is protected by a suitable selectively removable protecting group. A different, selectively removable protecting group is utilized for amino acids containing a reactive side group such as lysine.

The resultant exemplary linear peptides may then be reacted to form their corresponding cyclic peptides. A method for cyclizing peptides is described in Zimmer, et al., Peptides, 393-394 (1992), ESCOM Science Publishers, B.V., 1993. To cyclize peptides containing two or more cysteines through the formation of disulfide bonds, the methods described by Tam et al., J. Am. Chem. Soc., 113:6657-6662 (1991); Plaue, Int. J. Peptide Protein Res., 35:510-517 (1990); Atherton, J. Chem. Soc. Trans. 1:2065 (1985); B. Kamber, et al., Helv. Chim Acta 63:899 (1980) are useful in some embodiments. Polypeptide cyclization is a useful modification to generate modified peptides (e.g., peptidomimetics) because of the stable structures formed by cyclization and in view of the biological activities observed for cyclic peptides.

Alternatively, selected peptides that are useful in the present invention are produced by expression of recombinant DNA constructs prepared in accordance with well-known methods once the peptides are known. Such production can be desirable to provide large quantities or alternative embodiments of such compounds. Production by recombinant means may be more desirable than standard solid phase peptide synthesis for peptides of at least 8 amino acid residues. The DNA encoding the desired peptide sequence is preferably prepared using commercially available nucleic acid synthesis methods. Following these nucleic acid synthesis methods, DNA is isolated in a purified form which encodes the peptides. Methods to construct expression systems for production of peptides in recombinant hosts are also generally known in the art. Preferred recombinant expression systems, when transformed into compatible hosts, are capable of expressing the DNA encoding the peptides. Other preferred methods used to produce peptides comprise culturing the recombinant host under conditions that are effective to bring about expression of the encoding DNA to produce the peptide of the invention and ultimately to recover the peptide from the culture.

Expression can be effected in either procaryotic or eukaryotic hosts. Prokaryotes most frequently are represented by various strains of E. coli. However, other microbial strains may also be used, such as bacilli, for example Bacillus subtilis, various species of Pseudomonas, or other bacterial strains. In such procaryotic systems, plasmid vectors that contain replication sites and control sequences derived from a species compatible with the host are used. For example, a workhorse vector for E. coli is pBR322 and its derivatives. Commonly used procaryotic control sequences, which contain promoters for transcription initiation, optionally with an operator, along with ribosome binding-site sequences, include such commonly used promoters as the beta-lactamase (penicillinase) and lactose (lac) promoter systems, the tryptophan (trp) promoter system, and the lambda-derived PL promoter and N-gene ribosome binding site. However, any available promoter system compatible with procaryote expression can be used.

In one embodiment, the invention provides portions of IVS4 NQVDQVGY (SEQ ID NO:1) and portions of IS4 SRPQGPFL (SEQ ID NO:2) that are useful for reducing cell migration in vivo and/or in vitro, and for reducing one or more symptoms of pathological conditions and/or biochemical processes that involve MMP-9-induced cell migration. The term “portion” when used in reference to a protein (as in a “portion of a given protein”) refers to fragments of that protein. The fragments may range in size from an exemplary 4, 10, 20, 30, and/or 50 contiguous amino acid residues to the entire amino acid sequence minus one amino acid residue. Thus, a polypeptide sequence comprising “at least a portion of an amino acid sequence” comprises from four (4) contiguous amino acid residues of the amino acid sequence to the entire amino acid sequence.

In one embodiment, the portion of IVS4 NQVDQVGY (SEQ ID NO:1) is exemplified by NQVDQVG (SEQ ID NO: 7), NQVDQV (SEQ ID NO: 8), NQVDQ (SEQ ID NO: 9), NQVD (SEQ ID NO: 10), QVDQVGY (SEQ ID NO: 11), VDQVGY (SEQ ID NO: 12), DQVGY (SEQ ID NO: 13), QVGY (SEQ ID NO: 14), QVDQVG (SEQ ID NO: 15), VDQVG (SEQ ID NO: 16), DQVG (SEQ ID NO: 17), QVDQV (SEQ ID NO: 18), VDQV (SEQ ID NO: 19), and QVDQ (SEQ ID NO: 20).

In another embodiment, the portion of IS4 SRPQGPFL (SEQ ID NO: 2) is exemplified by SRPQGPF (SEQ ID NO: 21), SRPQGP (SEQ ID NO: 22), SRPQG (SEQ ID NO: 23), SRPQ (SEQ ID NO: 24), RPQGPFL (SEQ ID NO: 25), PQGPFL (SEQ ID NO: 26), QGPFL (SEQ ID NO: 27), GPFL (SEQ ID NO: 28), RPQGPF (SEQ ID NO: 29), PQGPF (SEQ ID NO: 30), QGPF (SEQ ID NO: 31), RPQGP (SEQ ID NO: 32), PQGP (SEQ ID NO: 33), and RPQG (SEQ ID NO: 34).

In one embodiment, the invention's compositions are pharmaceutical compositions. The terms “pharmaceutical” and “physiologically tolerable” composition refers to a composition that contains pharmaceutically acceptable molecules, i.e., molecules that are capable of administration to or upon a subject and that do not substantially produce an undesirable effect such as, for example, adverse or allergic reactions, dizziness, gastric upset, toxicity and the like, when administered to a subject. Preferably also, the pharmaceutically acceptable molecule does not substantially reduce the activity of the invention's compositions. Pharmaceutical molecules include, but are not limited to excipients and diluents.

An “excipient” is an inactive substance used as a carrier for the invention's compositions that may be useful for delivery, absorption, bulking up to allow for convenient and accurate dosage of the invention's compositions. Excipients include, without limitation, antiadherents, binders (e.g., starches, sugars, cellulose, modified cellulose such as hydroxyethyl cellulose, hydroxypropyl cellulose and methyl cellulose, lactose, sugar alcohols such as xylitol, sorbital and maltitol, gelatin, polyvinyl pyrrolidone, polyethylene glycol), coatings (e.g., shellac, corn protein zein, polysaccharides), disintegrants (e.g., starch, cellulose, crosslinked polyvinyl pyrrolidone, sodium starch glycolate, sodium carboxymethyl cellulosemethycellulose), fillers (e.g., cellulose, gelatin, calcium phosphate, vegetable fats and oils, and sugars, such as lactose), diluents, flavors, colors, glidants (e.g., silicon dioxide, talc), lubriants (e.g., talc, silica, fats, stearin, magnesium strearate, steaic acid), preservatives (e.g., antioxidants such as vitamins A, E, C, selenium, cystein, methionine, citric acids, sodium citrate, methyl papaben, propyl paraben), sorbents, sweetners (e.g., syrup). In one embodiment, the excipient comprises HEC (hydroxyethylcellulose), which is a nonionic, water-soluble polymer that can thicken, suspend, bind, emulsify, form films, stabilize, disperse, retain water, and provide protective colloid action. HEC is non-inflammatory and has been used as a delivery vehicle for vaginal microbicides (Tien et al., AIDS Research & Human Retroviruses, (2005). 21:845).

Exemplary “diluents” include water, saline solution, human serum albumin, oils, polyethylene glycols, aqueous dextrose, glycerin, propylene glycol or other synthetic solvents.

### B. Exemplary Uses of the Invention's Compositions

The invention's compositions may be used in a method for reducing one or more symptoms of disease in a subject, comprising a) providing i) a mammalian subject in need of reducing one or more symptoms of disease, and ii) any of the compositions disclosed herein that contain at least a portion of IVS4 (SEQ ID NO:1) and/or at least a portion of IS4 (SEQ ID NO:2), and b) administering to the subject a therapeutic amount of the composition to produce a treated subject, wherein the administering is under conditions for reducing one or more symptoms of the disease.

The term “administering” when in reference to a polypeptide, means providing the polypeptide to a subject. This may be done using methods known in the art (e.g., Erickson et al., U.S. Pat. No. 6,632,979; Furuta et al., U.S. Pat. No. 6,905,839; Jackobsen et al., U.S. Pat. No. 6,238,878; Simon et al., U.S. Pat. No. 5,851,789). The invention's compositions may be administered prophylactically (i.e., before the observation of disease symptoms) and/or therapeutically (i.e., after the observation of disease symptoms). Administration also may be concomitant with (i.e., at the same time as, or during) manifestation of one or more disease symptoms. Also, the invention's compositions may be administered before, concomitantly with, and/or after administration of another type of drug or therapeutic procedure (e.g., surgery). Methods of administering the invention's compositions include, without limitation, administration in parenteral, oral, intraperitoneal, intranasal, topical and sublingual forms. Parenteral routes of administration include, for example, subcutaneous, intravenous, intramuscular, intrasternal injection, and infusion routes.

In one embodiment, the invention's compositions comprise a lipid for delivery as liposomes. Methods for generating such compositions are known in the art (Borghouts et al. (2005). J Pept Sci 11, 713-726; Chang et al. (2009) PLoS One 4, e4171; Faisal et al. (2009) Vaccine 27, 6537-6545; Huwyler et al. (2008) Int J Nanomedicine 3, 21-29; Song et al. (2008) Int J Pharm 363, 155-161; Voinea et al. J Cell Mol Med 6, 465-474).

A “subject” and “animal” that may benefit from the invention's methods interchangeably includes any multicellular animal, preferably a mammal. Mammalian subjects include humans, non-human primates, murines, ovines, bovines, ruminants, lagomorphs, porcines, caprines, equines, canines, felines, ayes, etc.). Thus, mammalian subjects are exemplified by mouse, rat, guinea pig, hamster, ferret and chinchilla.

“Subject in need of reducing one or more symptoms of” a disease, e.g., in need of reducing cancer metastasis and/or in need of reducing one or more symptoms of cancer, includes a subject that exhibits and/or is at risk of exhibiting one or more symptoms of the disease. For Example, subjects may be at risk based on family history, genetic factors, environmental factors, etc. This term includes animal models of the disease. Thus, administering a composition (which reduces a disease and/or which reduces one or more symptoms of a disease) to a subject in need of reducing the disease and/or of reducing one or more symptoms of the disease includes prophylactic administration of the composition (i.e., before the disease and/or one or more symptoms of the disease are detectable) and/or therapeutic administration of the composition (i.e., after the disease and/or one or more symptoms of the disease are detectable).

The invention's compositions are administered to a subject in a theraprutically effective amount. As used herein the terms “therapeutically effective amount” and “protective amount” of a composition with respect to HIV infection refer to, in one embodiment, an amount of the composition that delays, reduces, palliates, ameliorates, stabilizes, prevents and/or reverses one or more symptoms of a disease, compared to in the absence of the composition of interest. Examples include, without limitation, tumor size and/or tumor number in cancer disease, glucose levels in blood and/or urine in diabetes, standard biochemical kidney function tests in kidney disease, etc. The term “delaying” symptoms refers to increasing the time period between exposure to the immunogen or virus and the onset of one or more symptoms of the exposure. The term “eliminating” symptoms refers to 100% reduction of one or more symptoms.

Specific dosages (i.e., amounts) that are encompassed by the “pharmaceutically effective amount,” “therapeutically effective amount” and “protective amount” can be readily determined by clinical trials and depend, for example, on the route of administration, patient weight (e.g. milligrams of drug per kg body weight), the type of subject being treated, and the physical characteristics of the specific subject under consideration. These factors and their relationship to determining this amount are well known to skilled practitioners in the medical, veterinary, and other related arts. This amount and the method of administration can be tailored to achieve optimal efficacy but will depend on such factors as weight, diet, concurrent medication and other factors which those skilled in the art will recognize. The dosage and frequency are selected to create an effective level of the compound without substantially harmful effects.

A pharmaceutically effective amount may be determined using in vitro and in vivo assays known in the art and disclosed herein.

Indeed, the invention's methods are useful in any disease that involves MMP-9-induced cell migration, such as migration of a cancer cell. In one embodiment, the disease is exemplified by cancer, cancer metastasis, systemic lupus erythematosus (SLE), Sjogren's syndrome (SS), systemic sclerosis (SS), polymyositis, rheumatoid arthritis (RA), multiple sclerosis (MS), atherosclerosis, cerebral ischemia, abdominal aortic aneurysm (AAA), myocardial infarction (MI), cerebral amyloid angiopathy (CAA), angiogenesis, inflammation, ectopic eczema, and contact eczema.

Cancer that may be ameliorated using the invention's methods and compositions include, for example, carcinomas such as lung cancer, breast cancer, prostate cancer, cervical cancer, pancreatic cancer, colon cancer, ovarian cancer; stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia. Malignant neoplasms are further exemplified by sarcomas (such as osteosarcoma and Kaposi's sarcoma). In a particular embodiment, the following cancer are preferred candidates for the invention's methods since MMP-9 has been found by DNA microarray data mining studies to be upregulated in these human cancers, including breast, brain and CNS, gastrointestinal, head and neck, kidney, lung, lymphoma, melanoma, ovarian cancers, sarcoma, neuroblastoma, and lymphoblastic cancer.

In another particular embodiment, the cancer cell is a metastatic cancer cell, cancer cell line (e.g. MCF-7, MDA-MB-231, MDA-435, HT-1080, LNCaP, DU145, PC3, TK4, C-1H, C-26, Co-3, HT-29, KM12SM, 253F B-V), etc.

In yet another embodiment, the invention's methods are useful in diseases that involve MMP-9-induced cell migration, such as migration of an endothelial cell, leukocyte cell (including neutrophils, dendritic cells, macrophages, eosinophils, mast cells, T lymphocytes, Langerhans' cells (LCs), etc.), fibroblast cell, osetoclast cell, osteoblast cell, etc.

Table 1 lists exemplary cells whose migration may be altered by the invention's compositions, thereby resulting in a reduction of one or more symptoms of the associated pathological condition.

TABLE 1
Examplary cells and pathological conditions

| Cell | Pathological condition |
|---|---|
| Cancer cells (including metastatic | Cancer and cancer metastasis (e.g., |
| cancer cells), cancer cell lines, | bjorklund et al. (2005) biochimica et |
| e.g., MCF-7, MDA-MB-231, | biophysica acta 1755: 37-69) |
| MDA-435, HT-1080, LNCaP, | |
| DU145, PC3, TK4, C-1H, C-26, | |
| Co-3, HT-29, KM12SM, | |
| 253F B-V | |
| Neutrophils, macrophages, T cells | Systemic lupus erythematosus (SLE) |
| White blood cells | Sjogren's syndrome (SS) |
| Fibroblasts | Systemic sclerosis (SS) |
| T lymphocytes | Polymyositis |
| Neutrophils, macrophages, T cells, | [symbol] d arthritis (RA) |
| osetoclasts, | |
| T cells, macrophages | Multiple sclerosis (MS) |
| Macrophages, T lymphocytes, | Atherosclerosis |
| endothelial cells | |
| Endothelial cells, macrophages | Cerebral ischemia |
| Macrophages | Abdominal aortic aneurysm (AAA) |
| Leukocytes, macrophages | Myocardial infarction (MI) |
| Endothelial cells, macrophages | Cerebral amyloid angiopathy (CAA) |
| Endothelial cell | Angiogenesis (Bjorklund et |
| | al. (2005)) |
| Leukocytes (including neutrophils, | Inflammation (Bjorklund |
| dendritic cells, macrophages, | et al. (2005)) |
| eosinophils, mast cells | |
| and lymphocytes) | |
| Leukocytes (including | Ectopic eczema, contact eczema |
| Langerhans' cells (LCs) | (Msika, patent application us |
| | 2004/0067910, filed aug. 25, 2003) |

[symbol]

indicates data missing or illegible when filed

The invention's methods may further comprise c) detecting a reduction in one or more symptoms of the disease in the treated subject. In one embodiment, the one or more symptoms of the disease comprise increased cell migration in the presence of MMP-9 compared to in the absence of MMP-9.

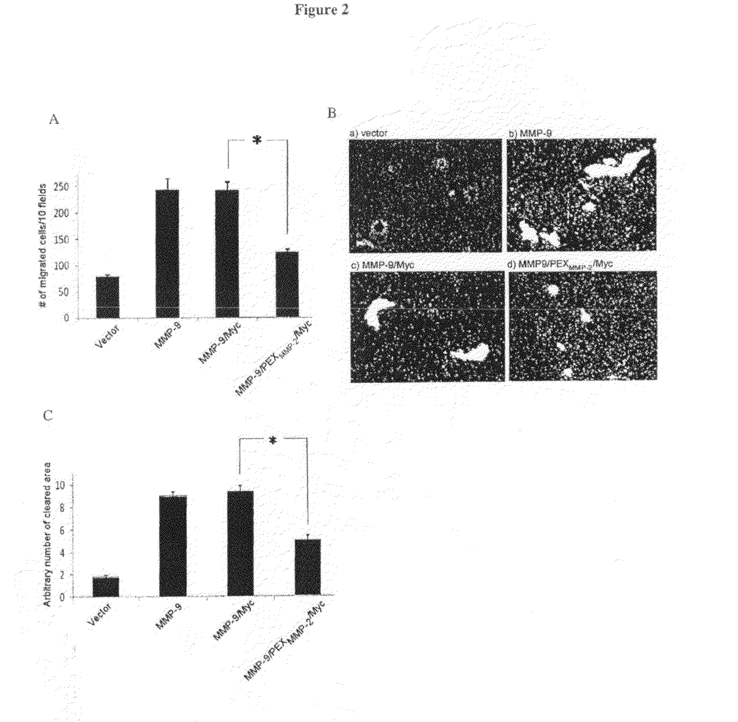

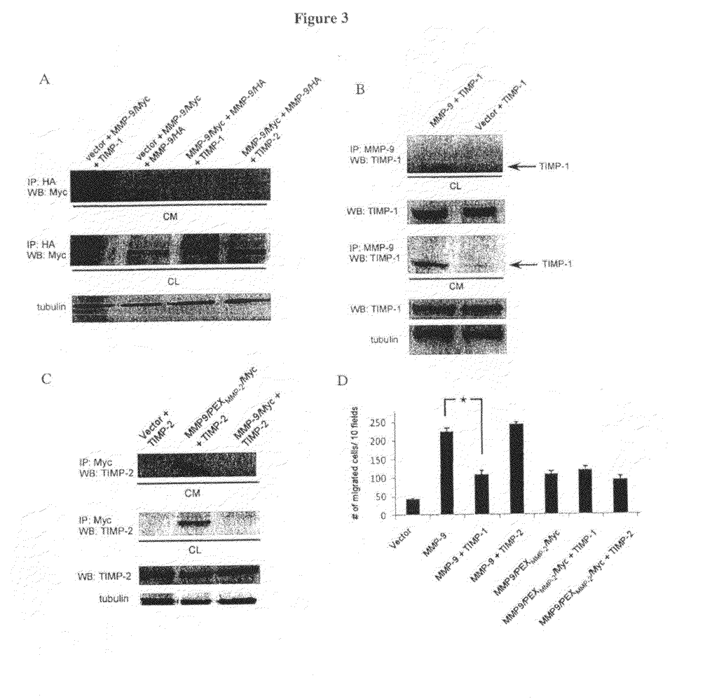

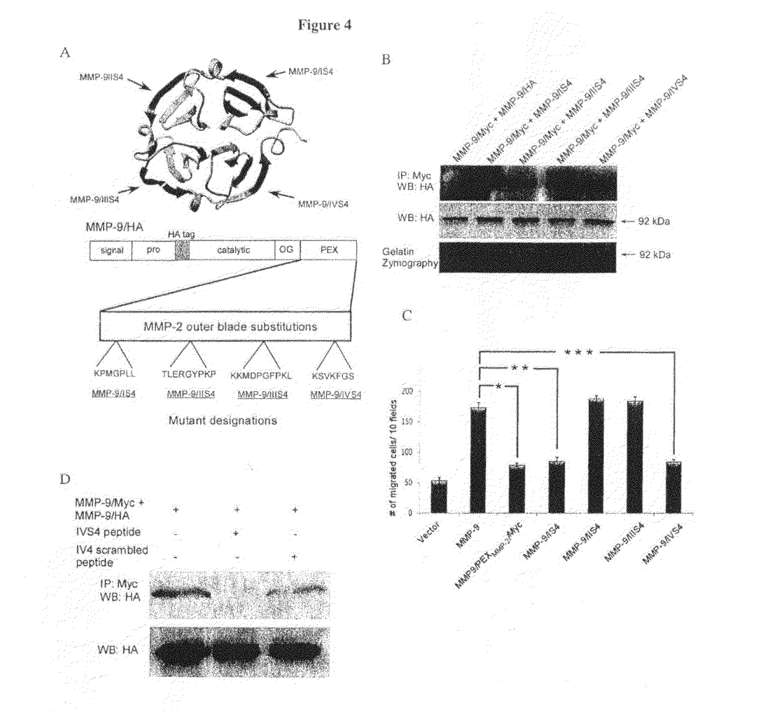

“Migration,” “migrating,” “motility” and grammatical equivalents when used in reference to a cell, interchangeably refer to the spatial movement of a cell on a 2-dimensional substrate (such as a solid substrate, or on a feeder layer of cells on a solid substrate), and/or within a 3-dimensional matrix (such as within a 3-dimensional collagen matrix). Methods for determining the level of cell migration are known in the art, such as wound induced migration assay (e.g., Ezhilarasan et al. (2009) Int. J. Cancer 125:306-315), and disclosed herein, such as transwell migration assay (Example 3, FIG. 2 & Example 4, FIG. 3)

In one embodiment, the therapeutic amount of the invention's composition specifically reduces the cell migration. The term “specifically reduces” when in reference to the level of a particular compound (e.g., the invention's polypeptides) and/or particular phenomenon (e.g., MMP-9-induced cell migration)” means the preferential reduction (i.e., a statistically significant reduction) in the level of the particular compound and/or particular phenomenon as compared to the level of another compound and/or phenomenon. For example, data herein demonstrate that the invention's polypeptides specifically reduce MMP-9-induced cell migration as compared to MMP-2-induced cell migration and/or MT1-MMP-induced cell migration.

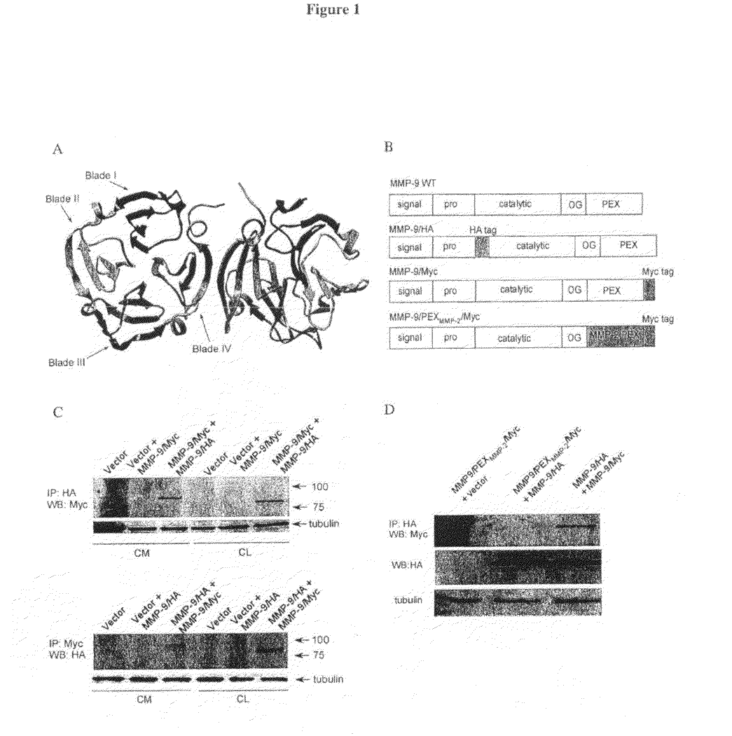

While not intending to limit the invention's IVS4, and portions thereof, to any particular mechanism, in one embodiment, the composition comprises an amount of at least a portion of NQVDQVGY (SEQ ID NO:1) (IVS4) that reduces homodimerization of MMP-9. Data herein demonstrate the effect of the invention's polypeptides on “homodimerization” of MMP-9 (Example 2, FIG. 1).

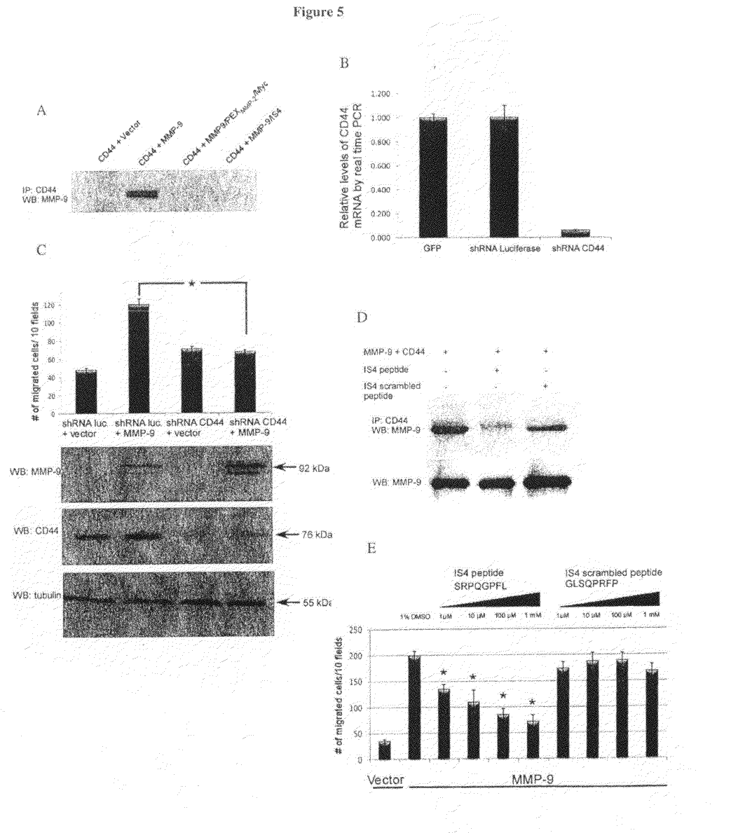

The term “homodimerization” refers to the oligomerization between two polypeptides having the same amino acid sequence. On the other hand, the terms “heterodimerization” refers to the oligomerization between two polypeptides having different amino acid sequences. An amino acid sequence is different from another amino acid sequence if it contains one or more amino acids that are not the same as the amino acids in the other amino acid sequence. Data herein demonstrate the effect of the invention's polypeptides on Data herein demonstrate the effect of the invention's polypeptides on “homodimerization” of MMP-9 (Example 2, FIG. 1) and on “heterodimerization” of MMP-9 and CD44 (Example 7, FIG. 5).

The term “specific oligomerization,” “specific binding,” “specific pairing,” “binding specificity,” and “pairing specificity,” when made in reference to two protein sequences is herein used to refer to the preferential oligomerization between two protein sequences as compared to the oligomerization between either of these two protein sequences to a third protein sequence. Specific oligomerization may be heterospecific or homospecific. The terms “homospecific oligomerization” and “homospecificity” as used herein refer to the specific oligomerization between two or more polypeptides having the same amino acid sequence. On the other hand, the terms “heterospecific oligomerization” and “hetero specificity” refer to the specific oligomerization between two or more polypeptides having different amino acid sequences.

Without limiting the invention's IS4, and portions thereof, to any particular mechanism, in one embodiment, the composition comprises an amount of at least a portion of SRPQGPFL (SEQ ID NO:2) (IS4) that reduces heterodimerization of MMP-9 and CD44. Data herein demonstrate the effect of the invention's polypeptides on “heterodimerization” of MMP-9 and CD44 (Example 7, FIG. 5).

### C. Discussion of the Exemplary Embodiments in Examples 1-8

Non-proteolytic activities of matrix metalloproteinases (MMPs) have recently been shown to impact cell migration, but the precise mechanism remains to be understood. The inventors previously demonstrated that the hemopexin (PEX) domain of MMP-9 is a prerequisite for enhanced cell migration. Using a biochemical approach, the invention provides the discovery that dimerization of MMP-9 through the PEX domain is necessary for MMP-9-enhanced cell migration. Following a series of substitution mutations within the MMP-9 PEX domain, blade IV was shown to be critical for homodimerization, whereas blade I was required for heterodimerization with CD44. Both blades I and IV mutants showed diminished enhancement of cell migration compared to wild type MMP-9 transfected cells. Peptides mimicking motifs in the outermost strands of the first and fourth blades of MMP-9 PEX domain were designed, which efficiently blocked MMP-9 dimer formations and inhibited motility of MMP-9 transfected cells. Using a shRNA approach, CD44 was found to be a critical molecule in MMP-9-mediated cell migration. Furthermore, an axis involving an MMP-9-CD44-EGFR signaling pathway in cell migration was identified using antibody array and specific receptor tyrosine kinase inhibitors. In conclusion, the inventors dissected the mechanism of proMMP-9-enhanced cell migration and developed structure-based inhibitory peptides targeting MMP-9-mediated cell migration.

The proteolytic activity of MMP-9 has been implicated in various physiologic and pathologic conditions. Inhibition of the catalytic domain has been a long-term focus of MMP research. More recently, the PEX domain has been demonstrated to be critical for mediating protein-protein interactions and enhancing cell migration (Dufour et al., 2008; Redondo-Munoz et al., 2008; Stefanidakis et al., 2009). The inventors demonstrated that the proteolytic activity of MMP-9 is not required for enhanced cell migration (Dufour et al., 2008). By swapping the MMP-9 PEX with that of MMP-2, the inventors herein demonstrate the unique homodimerization properties of the MMP-9 PEX domain. This chimeric substitution of the MMP-9 PEX domain in transfected COS-1 cells resulted in reduced cell migration.

The proteolytic activity of secreted MMPs is inhibited by TIMPs binding in a 1:1 ratio to the catalytic core domain. It has also been shown that the MMP-9 PEX domain binds specifically to TIMP-1, whereas the PEX domain of MMP-2 binds to TIMP-2 (Goldberg et al., 1992; Morgunova et al., 2002). Based on a crystallography analysis, TIMP-2 forms a complex with proMMP-2 through interaction with the blade IV of the PEX domain of MMP-2 (Morgunova et al., 2002). A precise interaction between the proMMP-9 PEX domain and TIMP-1, however, has not been solved by crystallography. Using a biochemical approach, the inventors herein demonstrate that TIMP-1 interacts with proMMP-9. This observation raises the question: can TIMP-1 competitively interfere with proMMP-9 homodimerization and hence, inhibit MMP-9 biological properties? Using a biochemical assay, the inventors show that TIMP-1 interfered with MMP-9 homodimerization either competitively, caused by overlapping contact areas, or allosterically, caused by conformational changes to PEX-9 that render it incompetent for homodimerization. This interaction reduced MMP-9-mediated cell migration.

Since TIMP-1 has been reported to form a complex with proMMP-9 prior to secretion (Roderfeld et al., 2007), the inventors tested their hypothesis that MMP-9 homodimer formation occurs intracellularly, which may stabilize MMP-9 during trafficking. Homodimer formation of MMP-9 features functions that are not present in monomeric PEX of MMP-9, such as electrostatic potential at the physico-chemical level (Cha et al., 2002). Moreover, an extended hydrophobic surface patch accessible to solvent in the monomeric MMP-9 PEX domain becomes buried upon dimerization. Finally, the dimeric complex brings all domains, including the catalytic domains, of the two MMP-9 monomers to within a defined distance from each other. This distance restraint is likely to influence a multitude of functions, including accessibility for proteolytic activation (Olson et al., 2000), localization to the extracellular matrix (Olson et al., 1998) and substrate recognition and processing (Collier et al., 2001).

Expression of MMP-9 in cells has been found to result in activation of MAPK and PI3K pathways; activation of these signaling molecules has been linked to the phosphorylation status of receptor tyrosine kinases (Ellerbroek et al., 2001; Yao et al., 2004). However, it remains to be explained how MMP-9 initiates signaling transduction leading to enhance cell migration. In order to pinpoint the signaling cascade of MMP-9 enhancement of cell migration, the inventors first screened the effect of inhibitors against different receptor tyrosine kinases on MMP-9-mediated cell migration. The inventors identified that phosphorylation of EGFR is involved in the MMP-9 signaling cascade. This observation was proven by four lines of evidence: 1) inhibition of EGFR (?) activation by specific inhibitors abrogates MMP-9-mediated cell migration; 2) activation of EGFR (?) in MMP-9 transfected cells was identified by antibody array analysis; 3) enhanced phosphorylation of EGFR (?) in MMP-9-transfected cells was confirmed by immunoblotting; and 4) downstream effectors of active EGFR (?), including pERK1/2, pAKT, pFAK, cofilin 1 and paxillin in MMP-9 expressing cells were upregulated or phosphorylated, as examined by antibody array or immunoblotting assays. The inventors' results agree with previous observation showing that inhibiting EGFR (?) activity with AG1478 in NIH 3T3 cells decrease phosphorylation of cofilin (Marcoux and Vuori, 2005).

It has been reported that CD44 and EGFR (?) interaction promotes cell migration involving activation of Akt, FAK and MMP-2 (Kim et al., 2008). In a separate report, Yu et al. (Yu and Stamenkovic, 1999) demonstrated that CD44 serves as a cell surface docking molecule for MMP-9. The inventors now present data showing that MMP-9 transduces signals to activate EGFR (?) through crosstalk with CD44 to initiate cell migration signaling cascade. This conclusion is based on the fact that silencing of CD44 in MMP-9 expressing cells abrogated activation of EGFR. MMP-9 and CD44 interactions, mediated through the outermost β-strand of blade I of the MMP-9 PEX domain, increased phosphorylation of EGFR which, in turn, activates FAK, AKT, ERK, cofilin 1 and paxillin leading to cell migration.

Docking of proMMP-9 at the cell surface by CD44 was reported to lead to activation of proMMP-9 in osteoclasts and result in enhanced cell migration (Samanna et al., 2007). In contrast, the inventors demonstrate that proteolytic activity of MMP-9 is not a prerequisite for MMP-9-mediated cell migration. Four lines of evidence support the inventors' previous conclusion that MMP-9-mediated COS-1 cell migration is independent of enzymatic activity (Dufour et al., 2008): 1) constitutively inactive MMP-9 (MMP-9^{E-A} mutation) induced cell migration in COS-1 cells as well as wild type MMP-9; 2) synthetic MMP inhibitors did not interfere with MMP-9-mediated COS-1 cell migration; 3) TIMP-1, but not TIMP-2 abrogates MMP-9-mediated cell migration; and 4) no activated MMP-9 is detected by fluorogenic peptide assay, gelatin zymography and western blotting. This discrepancy could be due to cell type differences, but the exact mechanism remains to be examined.

Targeting the PEX domain of MMP-9 has been the central focus in recent years since most MMP inhibitors blocking enzymatic activities failed to prove useful in several clinical trials (Coussens et al., 2002; Pavlaki and Zucker, 2003). Three approaches have been used to target the PEX domain of MMP-9: 1) antifunctional antibody: an anti-functional antibody targeting the MMP-9 PEX domain blocked Schwann cell migration (Mantuan et al., 2008); 2) recombinant protein of the PEX domain of MMP-9: Ezhilarasan et al. (Ezhilarasan et al., 2009) reported that excess recombinant MMP-9 PEX protein retarded endothelial cell migration; and 3) inhibitory peptides: Bjorklund et al., (Bjorklund et al., 2004) reported that random peptides isolated from a phage display peptide library inhibit cell migration and tumor formation by targeting either the MMP-9 catalytic domain or PEX domain. These studies emphasized the importance of the PEX domain of MMP-9 in cell migration and targeting this domain is a viable approach to abrogate pathologic cell migration. To date, no small molecular compounds have been developed for targeting the PEX domain of MMP-9. The inventors' strategy to develop synthetic compounds targeting the functional MMP-9 PEX domain is to identify targeting motifs within the PEX domain based on a mutagenesis approach followed by chemical synthesis. The inventors' peptides mimicking the outermost □-strand of blades I and IV of the MMP-9 PEX domain inhibited MMP-9-mediated cell migration in a dose-dependent manner. The core structure of these peptides is being evaluated by NMR and chemical synthesis will be followed.

In summary, data herein demonstrate a novel axis of MMP-9-CD44-EGFR in which MMP-9 initiates crosstalk between CD44 and EGFR, which in turn activates downstream effectors for cell migration. The inventors further pinpoint two critical motifs in the PEX domain of MMP-9 required for cell migration. The synthetic inhibitory peptides mimicking these motifs demonstrate that developing pharmaceutical compounds targeting these regions is a useful approach to impair cell migration during pathological processes.

### EXPERIMENTAL

The following examples serve to illustrate certain embodiments and aspects of the present invention and are not to be construed as limiting the scope thereof.

### Example 1

### Material and Methods

Reagents

Oligo primers were purchased from Operon (Al, Huntsville). The pcDNA3.1-myc expression vectors were purchased from Invitrogen (Carlsbad, Calif.). Anti-Myc, anti-HA antibodies were purchased from Roche (Indianapolis, Ind.). MMP-9 antibody was described previously (Dufour et al., 2008). Anti-tubulin, anti-AKT, anti-pAKT, anti-ERK, anti-pERK, anti-pEGFR and anti-EGFR antibodies were purchased from Cell Signaling Technology (Davers, Mass.). Anti-FAK and anti-pFAK antibodies were purchased from Bio Source (Camarillo, Calif.). Anti-TIMP-1 and anti-TIMP-2 antibodies were purchased from Calbiochem (Cambridge, Mass.). Anti-CD44 antibodies were purchased from Novas Biologicals (Littleton, Colo.).

Cell Culture and Transfection

COS-1 monkey kidney epithelial cell line was purchased from ATCC (Manassas, Va.) and was maintained in Dulbecco's modified Eagle's medium (Invitrogen). Transfection of plasmid DNA in COS-1 cells was achieved using polyethylenimine (Polysciences) or Transfectin™ reagents (Bio-Rad) and the transfected cells were incubated for 48 h at 37° C. followed by biochemical and biological assays.

Construction of Plasmids

MMP-9 with a carboxy-terminal Myc tag (MMP-9/Myc) was generated by using the pcDNA3.1 expression vector (Invitrogen). The MMP-9 cDNA containing the open reading frame of MMP-9 was amplified by a PCR approach using the primers sets: forward primer, #1315:5′-3′: CGGAATTCCGCCAACATGAGCCTCTGGCAGCCCCT (SEQ ID NO: 36) and reverse primer, #2507: 5′-3′ GGAAGATCTCTAGTCCTCAGGGCACTGCAGGATGTC (SEQ ID NO: 37). The resultant PCR fragment was then cloned into the pcDNA3.1 vector to generate MMP-9/Myc chimeric cDNA.

To generate HA (human influenza hemagglutinin)-tagged MMP-9 chimeric cDNA, the HA tag was placed between the propeptide and catalytic domains of MMP-9 by a site-directed mutagenesis approach using wild type MMP-9 as a template with mutagenesis primers containing HA sequence (forward primer, #2512: 5′-3′: GGGGTCCCAGACCTGGG CAGATACCCCTACGACGTGCCCGACTCGCCTCCAAACCTTTGAGGGCGAC (SEQ ID NO: 38) and reverse primer, #2513: 5′-3′:GTCGCCCTCAAAGGTTTGGAAGGCGTAGTCGGGCACGTCGTAGG GGTATCTGCCCAGGTCTGGGACCCC (SEQ ID NO: 39)) (QuickChange Site Directed Mutagenesis kit, Stratagene).

To determine the role of the PEX domain of MMP-9, a substitution mutation was engineered by replacing the PEX domain of MMP-9 with that of MMP-2. A modified two-step PCR was employed as previously described (Cao et al., 1998). In brief, MMP-9 signal peptide/propeptide/catalytic/hinge domains (fragment A) (primer sets: forward primer #1315 and reverse primer #2534, 5′-3′: TACAATGTCCTGTTTGCAGATCTCGTCCACCGGACTCAA AGGCAC) (SEQ ID NO: 40), and MMP-2 PEX domain (fragment B) (primer sets: forward primer #2535, 5′-3′: GAGATCTGCAAACAGGACATTGTATTTGAT (SEQ ID NO: 41), and reverse primer #1356, 5′-3′: CCCAAG CTTCTAGCAGCCTAGCCAGTCGGATTT) (SEQ ID NO: 42) were first amplified by PCR respectively. Using the resultant PCR products as templates (fragment A and B), the signal/propeptide/catalytic/hinge region of MMP-9 were then fused together with MMP-2 PEX domain by PCR using forward primer #1315 and reverse primer #1356. The resultant PCR fragments were then inserted into the pcDNA3.1 vector (invitrogen) to generate MMP9/PEX_{MMP2}/Myc chimeric cDNAs. Similarly, the inventors generated substitution mutations of MMP-9/Blade I, II, III and IV by replacing the outermost β-strand of each blade with the corresponding sequence of MMP-2. Primers used to generate these chimeric cDNAs were designed as follow: 1) MMP-9/Blade I, fragment A: #1315, and #2557 (5′-3′: GGGAGCCGG CCGGGGCCCCTGCTGATCGCCGAC (SEQ ID NO: 43); and fragment B: #2558 (5′-3′: GTCGGCGATCAGC AGGGGCCCCGGCCGGCTCCC) (SEQ ID NO: 44) and #2507; 2) MMP-9/Blade II, Fragment A: #1315, and #2596 (5′-3′: GTGTACACAGGCGCGACCTTGGAGCGAGGGTACCCCAAGCCACTGGA CAAGCTGGGC) (SEQ ID NO: 45); and fragment B: #2597 (5′-3′:CACATGTGTCCGCGCTGGAACCTCGC TCCCATGGGGTTCGGTGACCTGTTCGACCCG) (SEQ ID NO: 46), and #2507; 3) MMP-9/Blade III, fragment A: #1315, and #2540 (5′-3′: GTTCGACGTGAAGGCGAAGAAAATGGATCCTGGCTTCC CCAAGCTCGTGGACCGGATGTTCC) (SEQ ID NO: 47); and fragment B: #2541 (5′-3′:GGAACATCCGG TCCACGAGCTTGGGGAAGCCAGGATCCATTTTCTTCGCCTTCACGTCGAAC) (SEQ ID NO: 48), and #2507; and 4) MMP-9/Blade IV: fragment A: #1315, and #2538 (5′-3′: GTTCCCGGAGTG AGTTGAAGAGCGTGAAGTTTGGAAGCGTGACCTATGACATCCTG) (SEQ ID NO: 49); and fragment B: #2539 (5′-3′: CAGGATGTCATAGGTCACGCTTCCAAACTTCACGCTCTTCAACTCACT CCGGGAAC) (SEQ ID NO: 50), and #2507. All the constructs were confirmed by DNA sequencing.

Construction of Short Hairpin RNA Vectors and Retroviral Infection

Small interfering oligonucleotides specific for human and monkey CD44 and control luciferase to express short hairpin RNA were designed using a Worldwide Web-based online software system (Block-iT RNAi Designer, Invitrogen) for mammalian RNA interference. Two specific 21-nucleotide sequences spanning positions 173-193 (CD44shRNA-1) and 678-698 (CD44-shRNA-2) of the human CD44 gene (Gen-Bank accession number L05424) were synthesized. The sense and antisense template oligonucleotides encoding a hairpin structure were annealed and cloned into the RNAi-Ready pSIREN-Retro Q vector (Clontech). As a control, a luciferase protein from firefly Pyrocoelia pectoralis as a target gene was employed as previously reported (Cao et al., 2008).

A retroviral supernatant was obtained by co-transfection of a vector encoding the envelope gene (pAmphotropic) and a retroviral expression vector containing the CD44 shRNA, or luciferase shRNA control into human embryonic kidney GP2-293 packaging cells (Clontech) according to the manufacturer's protocol. COS-1 cells were infected with the viral supernatant, and the cells were then selected with 4 μg/ml puromycin for 1-2 weeks. The effects of shRNA on gene expression were evaluated by real time RT-PCR using RNA of pooled resistant cells. The most effective stable CD44 knockdown cell lines were selected.

Immunofluorescence

Cultured cells were fixed with 4% paraformaldehyde (PFA)/phosphate-buffered saline (PBS) followed by blocking with 3% bovine serum albumin (BSA)/PBS. CD44 was detected with anti-CD44 antibody (Novus Biologicals, CO) followed by secondary antibodies conjugated with Alexa 568 (Invitrogen). Nuclei were counterstained with DAPI (Invitrogen).

Flow Cytometry

1×10⁶ cells/ml cells suspended in Dulbecco's modified Eagle's medium containing 2% BSA were incubated with CD44 primary monoclonal antibody for 60 min at 4° C. followed by incubation with anti-mouse FITC labeled (1:1000) secondary antibody for 60 min at 4° C. After extensively washing, the cells were stained with propidium iodide (PI) to determine viability of the cells. CD44 expression was measured using a FACS Calibur flow cytometer (Becton Dickinson, San Jose, Calif.).

Antibody Microarray and Protein Phosphorylation Assay (Kinex™ Antibody Microarray KAM-1.2 and Phospho-Site 1.3 Kinetworks Screen)

Cell lysates were prepared as recommended by Kinexus Bioinformatics Corporation (Vancouver, BC, Canada). COS-1 cells were transiently transfected with vector or MMP-9 cDNA. Forty-eight hours after transfection, total cellular proteins were extracted using ice-cold lysis buffer containing detergent and protease inhibitor cocktail and subjected to a customer antibody microarray analysis (Kinexus Bioinformatics Corporation). Over 500 pan-specific and 300 phospho-site specific antibodies were screened against both vector control and MMP-9 samples. Sixteen different proteins which are known to link cell migration and displayed over two-fold difference between control and MMP-9 transfected cells were further analyzed by a semi-quantitative immunoblotting examination (Kinexus Bioinformatics Corporation).

Statistical Analysis

Data is expressed as the mean±standard error of triplicates. Each experiment was repeated as least 3 times. Student's t-test and analysis of variants (ANOVA) were used to assess differences with p<0.05 considered to be significant.

Procedures for Gelatin Substrate Zymography, Immunoblotting, Immunoprecipitation, Transwell Chamber Cell Migration and Phagokinetic Cell Migration Assays

Basic protocols for these techniques have been described previously (Cao et al., 1996; Dufour et al., 2008).

### Example 2

### Dimerization of MMP-9 in Transfected COS-1 Cells

The inventors previously demonstrated that the hemopexin (PEX) domain of proMMP-9 is required for the enhanced cell migration; this effect is independent of MMP-9 proteolytic activity (Dufour et al., 2008). In order to dissect the mechanism underlying MMP-9-induced cell migration, the PEX domain of MMP-9 was examined using biochemical and molecular approaches.

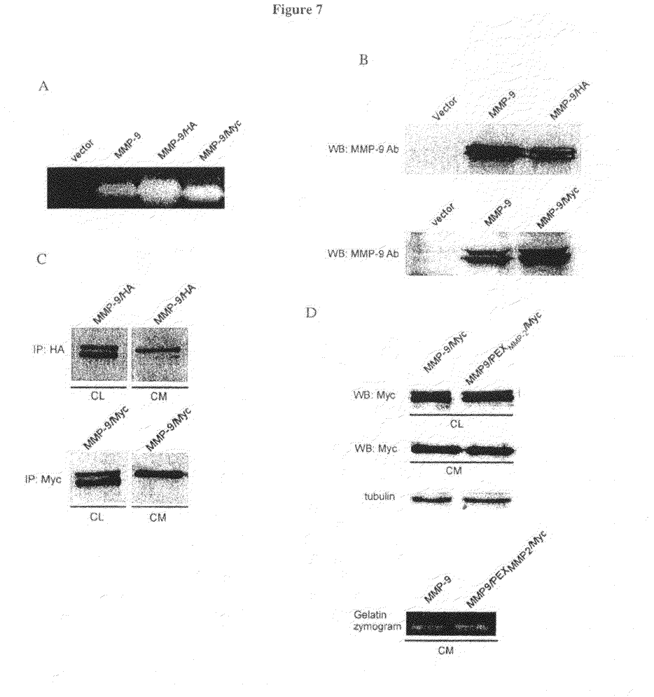

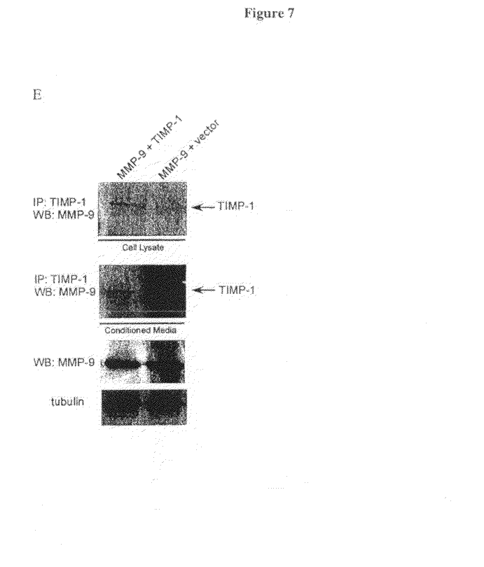

Among all secreted MMPs, only MMP-9 has been found to form a homodimer Employing proteins purified from an E. coli expression system (FIG. 1A, ribbon diagram based on PDB file: HTV), Cha et al. (Cha et al., 2002) showed that MMP-9 homodimer formation requires an interaction of the fourth blade of adjacent PEX domains Since proteins purified from a bacterial system lack posttranslational modifications, e.g. glycosylation and phosphorylation, which can impact the function of most mammalian proteins (Davis, 2004), it is essential to test if the dimerization of MMP-9 also occurs in a mammalian cell expression system. To this end, the inventors fused HA and Myc tags into the MMP-9 cDNA, to generate MMP-9/HA and MMP-9/Myc chimeras, respectively (FIG. 1B), followed by transfection of the cDNAs into COS-1 monkey kidney epithelial cells. Insertion of either HA tag between the propeptide domain and catalytic domain or Myc tag at the end of the PEX domain of MMP-9 does not interfere with the overall properties of wild type MMP-9 as evidenced by gelatin zymography and western blotting (FIGS. 7A and 7B). Immunoprecipitation with either HA or Myc antibodies resulted in identifying MMP-9 in both the cell lysates and conditioned media of transfected COS-1 cells (FIG. 7C).

COS-1 cells co-transfected with HA- and Myc-tagged MMP-9 cDNAs were then employed to examine homodimer formation using a co-immunoprecipitation approach. The HA-tagged MMP-9 proteins in the conditioned medium of transfected COS-1 cells were immunoprecipitated with anti-HA antibodies followed by a western blotting probed by anti-Myc antibodies. This approach revealed the identification of Myc-tagged MMP-9 in the complex immunoprecipitated by anti-HA antibody, suggesting that MMP-9 forms homodimers in cell condition medium (FIG. 1C). To further confirm this observation, the reciprocal co-immunoprecipitation was performed. Anti-Myc antibodies were employed to precipitate Myc-tagged MMP-9 complexes in the conditioned medium of co-transfected COS-1 cells followed by western blotting using anti-HA antibodies. This reciprocal approach confirms that MMP-9 does form a homodimer in the condition medium of transfected COS-1 cells. To further explore if homodimer formation of MMP-9 occurs within the cell or following secretion from the cell, the cell lysate of transfected COS-1 cells was also examined by the co-immunoprecipitation method (FIG. 1C). homodimer formation was demonstrated in the cell lysate, suggesting that MMP-9 dimerization occurs during MMP-9 secretion.

Results:

The inventors previously demonstrated that the hemopexin (PEX) domain of proMMP-9 is required for the enhanced cell migration; this effect is independent of MMP-9 proteolytic activity (Dufour et al., 2008). In order to dissect the mechanism underlying MMP-9-induced cell migration, the PEX domain of MMP-9 was examined using biochemical and molecular approaches.

Among all secreted MMPs, only MMP-9 has been found to form a homodimer. Employing proteins purified from an E. coli expression system (FIG. 1A, ribbon diagram based on PDB file: HTV), Cha et al. (Cha et al., 2002) showed that MMP-9 homodimer formation requires an interaction of the fourth blade of adjacent PEX domains. Since proteins purified from a bacterial system lack posttranslational modifications, e.g. glycosylation and phosphorylation, which can impact the function of most mammalian proteins (Davis, 2004), it is essential to test if the dimerization of MMP-9 also occurs in a mammalian cell expression system. To this end, the inventors fused HA and Myc tags into the MMP-9 cDNA, to generate MMP-9/HA and MMP-9/Myc chimeras, respectively (FIG. 1B), followed by transfection of the cDNAs into COS-1 monkey kidney epithelial cells. Insertion of either HA tag between the propeptide domain and catalytic domain or Myc tag at the end of the PEX domain of MMP-9 does not interfere with the overall properties of wild type MMP-9 as evidenced by gelatin zymography and western blotting (FIGS. 7A and 7B). Immunoprecipitation with either HA or Myc antibodies resulted in identifying MMP-9 in both the cell lysates and conditioned media of transfected COS-1 cells (FIG. 7C). COS-1 cells co-transfected with HA- and Myc-tagged MMP-9 cDNAs were then employed to examine homodimer formation using a co-immunoprecipitation approach. The HA-tagged MMP-9 proteins in the conditioned medium of transfected COS-1 cells were immunoprecipitated with anti-HA antibodies followed by a western blotting probed by anti-Myc antibodies. This approach revealed the identification of Myc-tagged MMP-9 in the complex immunoprecipitated by anti-HA antibody, suggesting that MMP-9 forms homodimers in cell condition medium (FIG. 1C). To further confirm this observation, the reciprocal co-immunoprecipitation was performed. Anti-Myc antibodies were employed to precipitate Myc-tagged MMP-9 complexes in the conditioned medium of co-transfected COS-1 cells followed by western blotting using anti-HA antibodies. This reciprocal approach confirms that MMP-9 does form a homodimer in the condition medium of transfected COS-1 cells. To further explore if homodimer formation of MMP-9 occurs within the cell or following secretion from the cell, the cell lysate of transfected COS-1 cells was also examined by the co-immunoprecipitation method (FIG. 1C). MMP-9 homodimer formation was demonstrated in the cell lysate, suggesting that MMP-9 dimerization occurs during MMP-9 secretion.

### Example 3

Prerequisite of MMP-9 dimerization in proMMP-9-induced cell migration The inventors previously demonstrated that the PEX domain of proMMP-9 plays an important role in enzymatic activity-independent cell migration (Dufour et al., 2008). To test if dimerization of the MMP-9 PEX domain is a prerequisite for MMP-9-induced cell migration, the inventors generated MMP-9 PEX domain mutations. Since MMP-2 does not form homodimers (Morgunova et al., 2002), the PEX domain of MMP-9 was replaced by that of MMP-2 to generate MMP9/PEX_{MMP2} chimeric cDNA using a two-step PCR approach (Cao et al., 1996). The resultant PCR product containing MMP9/PEX_{MMP2} was then inserted into a pcDNA3.1/Myc vector to generate a final construct of MMP9/PEX_{MMP-2}/Myc chimeric cDNA (FIG. 1B). Substitution of the PEX domain of MMP-9 with that of MMP-2 failed to couple with the wild type MMP-9 in co-transfected COS-1 cells not only in the conditioned medium (FIG. 1D), but also in the cell lysate. The failure of the dimerization of the PEX domain mutation was not due to the loss of protein synthesis, trafficking, or proteolytic activity as evidenced by western blotting and gelatin zymography (FIG. 7D).

In order to determine if the loss of homodimerization of this MMP-9 PEX domain mutation affects MMP-9-induced cell migration, a transwell chamber migration assay was employed (Dufour et al., 2008). COS-1 cells transfected with the PEX mutant MMP-9 (MMP-9/PEX_{MMP-2}/Myc) failed to enhance cell migration to the extent of wild type MMP-9, suggesting a critical role of MMP-9 homodimer in cell migration (FIG. 2A). To further confirm the biological role of MMP-9 homodimers in cell migration, wild-type and mutant MMP-9-transfected cells were subjected to phagokinetic migration analysis which permits quantification by clearance of colloidal gold particles within the cell migratory path (FIG. 2B). Similarly to the data from the transwell migration assay (FIG. 2A), cells transfected with MMP-9/Myc cDNA displayed enhanced migration as compared to vector-transfected cells determined by the NIH ImageJ software (FIG. 2C). Enhanced cell migration did not occur in cells transfected with MMP-9/PEX_{MMP-2}/Myc. Taken together, these data indicate that homodimerization through the MMP-9 PEX domain plays a critical role in MMP-9-induced cell migration.

Results

The inventors previously demonstrated that the PEX domain of proMMP-9 plays an important role in enzymatic activity-independent cell migration (Dufour et al., 2008). To test if dimerization of the MMP-9 PEX domain is a prerequisite for MMP-9-induced cell migration, the inventors generated MMP-9 PEX domain mutations. Since MMP-2 does not form homodimers (Morgunova et al., 2002), the PEX domain of MMP-9 was replaced by that of MMP-2 to generate MMP9/PEX_{MMP2} chimeric cDNA using a two-step PCR approach (Cao et al., 1996). The resultant PCR product containing MMP9/PEX_{MMP-2} was then inserted into a pcDNA3.1/Myc vector to generate a final construct of MMT9/PEX_{MMP-2}/Myc chimeric cDNA (FIG. 1B). Substitution of the PEX domain of MMP-9 with that of MMP-2 failed to couple with the wild type MMP-9 in co-transfected COS-1 cells not only in the conditioned medium (FIG. 1D), but also in the cell lysate. The failure of the dimerization of the PEX domain mutation was not due to the loss of protein synthesis, trafficking, or proteolytic activity as evidenced by western blotting and gelatin zymography (FIG. 7D).

In order to determine if the loss of homodimerization of this MMP-9 PEX domain mutation affects MMP-9-induced cell migration, a transwell chamber migration assay was employed (Dufour et al., 2008). COS-1 cells transfected with the PEX mutant MMP-9 (MMP-9/PEX_{MMP-2}/Myc) failed to enhance cell migration to the extent of wild type MMP-9, suggesting a critical role of MMP-9 homodimer in cell migration (FIG. 2A). To further confirm the biological role of MMP-9 homodimers in cell migration, wild-type and mutant MMP-9-transfected cells were subjected to phagokinetic migration analysis which permits quantification by clearance of colloidal gold particles within the cell migratory path (FIG. 2B). Similarly to the data from the transwell migration assay (FIG. 2A), cells transfected with MMP-9/Myc cDNA displayed enhanced migration as compared to vector-transfected cells determined by the NIH ImageJ software (FIG. 2C). Enhanced cell migration did not occur in cells transfected with MMP-9/PEX_{MMP-2}/Myc. Taken together, these data indicate that homodimerization through the MMP-9 PEX domain plays a critical role in MMP-9-induced cell migration.

### Example 4

### Inhibition MMP-9 Dimerization by TIMP-1 Resulting in Diminished Cell Migration

The role of TIMP-1 in interfering with MMP-9 homodimer formation remains controversial (Goldberg et al., 1992; Olson et al., 2000). This discrepancy led us to reevaluate the specific interference of TIMP-1 on proMMP-9 homodimer formation using the biochemical approach described above. Since MMP-9 homodimer formation is a prerequisite for enhancing cell migration (FIG. 2A) and TIMP-1 blocks MMP-9-induced cell migration (Dufour et al., 2008), the inventors hypothesized that TIMP-1 binding to the PEX domain, might act as an inhibitor of proMMP-9 homodimerization, thus, interfering with MMP-9-induced cell migration. To test this hypothesis, COS-1 cells transfected with both proMMP-9/Myc and proMMP-9/HA cDNAs along with TIMP-1 or TIMP-2 cDNAs were examined by the co-immunoprecipitation assay, as described above. As shown in FIG. 3A, co-expression of TIMP-1 with proMMP-9 cDNAs resulted in blocking MMP-9 homodimer formation both in the conditioned media and cell lysate of transfected cells. As demonstrated by co-immunoprecipitation studies, abrogation of proMMP-9 homodimer formation by TIMP-1 coincided with heterodimer formation between proMMP-9 and TIMP-1 (FIGS. 3B & 7E). In contrast, TIMP-2 has no effect on proMMP-9 homodimer formation (FIG. 3A), which is in agreement with previous observation that TIMP-2 only bound to the PEX domain of MMP-2 but not MMP-9 (FIG. 3C) (Morgunova et al., 2002). Since TIMP-1 was shown to interfere with MMP-9 homodimerization, the inventors then tested if the loss of homodimer formation of MMP-9 by TIMP-1 fails to enhance cell migration. By employing a transwell migration assay, the inventors observed that co-expression of TIMP-1, but not TIMP-2, with proMMP-9 in COS-1 cells inhibited proMMP-9-enhanced cell migration, but had no apparent effect on proMMP9/PEX_{MMP-2}/Myc transfected cells (FIG. 3D). In addition, both TIMP-1 and TIMP-2 did not significantly inhibit MMP9/PEX_{MMP-7}/Myc enhancement of COS-1 cell migration (FIG. 3D). These results suggest a unique role for TIMP-1 interference of both MMP-9 homodimerization and enhancement of cell migration.

Results

The role of TIMP-1 in interfering with MMP-9 homodimer formation remains controversial (Goldberg et al., 1992; Olson et al., 2000). This discrepancy led us to reevaluate the specific interference of TIMP-1 on proMMP-9 homodimer formation using the biochemical approach described above. Since MMP-9 homodimer formation is a prerequisite for enhancing cell migration (FIG. 2A) and TIMP-1 blocks MMP-9-induced cell migration (Dufour et al., 2008), the inventors hypothesized that TIMP-1 binding to the PEX domain, might act as an inhibitor of proMMP-9 homodimerization, thus, interfering with MMP-9-induced cell migration. To test this hypothesis, COS-1 cells transfected with both proMMP-9/Myc and proMMP-9/HA cDNAs along with TIMP-1 or TIMP-2 cDNAs were examined by the co-immunoprecipitation assay, as described above. As shown in FIG. 3A, co-expression of TIMP-1 with proMMP-9 cDNAs resulted in blocking MMP-9 homodimer formation both in the conditioned media and cell lysate of transfected cells. As demonstrated by co-immunoprecipitation studies, abrogation of proMMP-9 homodimer formation by TIMP-1 coincided with heterodimer formation between proMMP-9 and TIMP-1 (FIGS. 3B & 7E). In contrast, TIMP-2 has no effect on proMMP-9 homodimer formation (FIG. 3A), which is in agreement with previous observation that TIMP-2 only bound to the PEX domain of MMP-2 but not MMP-9 (FIG. 3C) (Morgunova et al., 2002).

Since TIMP-1 was shown to interfere with MMP-9 homodimerization, the inventors then tested if the loss of homodimer formation of MMP-9 by TIMP-1 fails to enhance cell migration. By employing a transwell migration assay, the inventors observed that co-expression of TIMP-1, but not TIMP-2, with proMMP-9 in COS-1 cells inhibited proMIMP-9-enhanced cell migration, but had no apparent effect on proMMP9/PEX_{MMP-2}/Myc transfected cells (FIG. 3D). In addition, both TIMP-1 and TIMP-2 did not significantly inhibit MMP9/PEX_{MMP-2}/Myc enhancement of COS-1 cell migration (FIG. 3D). These results suggest a unique role for TIMP-1 interference of both MMP-9 homodimerization and enhancement of cell migration.

### Example 5

### Minimal Motif Required for MMP-9 Homodimer Formation

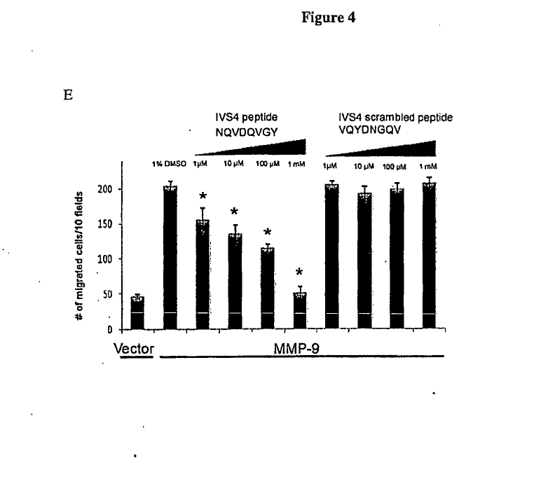

The PEX domain of MMPs exhibits similar structures composed of disc-like shape, with the chain folded into a β-propeller structure that has a pseudo four-fold symmetry. Each blade contains four anti-parallel □-strands with peptide loops linking one strand to the next as illustrated in FIG. 1B. Based on the crystal structure analysis of purified MMP-9 PEX domain from E. coli (Cha et al., 2002), the homodimerization of MMP-9 occurs through an interaction of the outermost strand of the fourth blade of the PEX domains. However, the crystal structure of MMP-9 PEX domain using the recombinant protein from E. coli has not been completely validated in a mammalian cell system. To test if the interaction interface of MMP-9 homodimer formation occurs through the outermost strand of the fourth blade of the PEX domains in mammalian cells, the inventors employed a genetic approach to generate substituted mutations for the outermost β-strands of each blade within the MMP-9 PEX domain. The outermost β-strand of the fourth blade of the MMP-9 PEX domain (N₆₈₈QVDQVGY₆₉₅) (SEQ ID NO: 1) was substituted by the corresponding region from MMP-2 PEX domain (K₅₈₆SVKFGS₅₉₂) (SEQ ID NO: 55) to generate MMP-9/IVS4 chimera (FIGS. 1A & 4A). As controls, three additional mutations for the outermost strands of blades I, II and III were engineered by replacing with the corresponding sequence of MMP-2 (MMP-9/IS4, MMP-9/IIS4 and MMP-9/IIIS4) (FIG. 4A). Employing a co-immunoprecipitation assay for COS-1 cells co-transfected with different combination of cDNAs, the inventors observed that mutations of blades I, II and III of MMP-9 PEX domain had no effect on MMP-9 homodimer formation; whereas mutation of blade IV (MMP-9/IVS4) failed to dimerize (FIG. 4B). This defect was not due to the loss of secretory ability of the mutant as evidenced by western blot and gelatin zymography of the conditioned medium from transfected COS-1 cells (FIG. 4B). The four mutants and wild type MMP-9 were then evaluated for their ability to enhance cell migration using a transwell migration assay (FIG. 4C). Mutation of the fourth β-strand of the PEX domain of MMP-9 (MMP-9/IVS4) failed to enhance COS-1 cells migration (FIG. 4C), as compared to wild type MMP-9, whereas mutations of blade II and III induced cell migration as well as wild type MMP-9. Unexpectedly, mutation of the first blade of the PEX domain of MMP-9 (MMP-9/1S4) also failed to induce cell migration. These data suggest that both blades I and IV of the MMP-9 PEX domain are required for the enhanced cell migration. Since the outermost β-strand of blade I is not involved in homodimer formation, another explanation for failure of migration of the MMP-9/IS4 mutant needs to be sought.

Results:

The PEX domain of MMPs exhibits similar structures composed of disc-like shape, with the chain folded into a β-propeller structure that has a pseudo four-fold symmetry. Each blade contains four anti-parallel □-strands with peptide loops linking one strand to the next as illustrated in FIG. 1B. Based on the crystal structure analysis of purified MMP-9 PEX domain from E. coli (Cha et al., 2002), the homodimerization of MMP-9 occurs through an interaction of the outermost strand of the fourth blade of the PEX domains. However, the crystal structure of MMP-9 PEX domain using the recombinant protein from E. coli has not been completely validated in a mammalian cell system. To test if the interaction interface of MMP-9 homodimer formation occurs through the outermost strand of the fourth blade of the PEX domains in mammalian cells, the inventors employed a genetic approach to generate substituted mutations for the outermost β-strands of each blade within the MMP-9 PEX domain. The outermost β-strand of the fourth blade of the MMP-9 PEX domain (N₆₈₈QVDQVGY₆₉₅) (SEQ ID NO: 1) was substituted by the corresponding region from MMP-2 PEX domain (K₅₈₆SVKFGS₅₉₂) (SEQ ID NO: 55) to generate MMP-9/IVS4 chimera (FIGS. 1A & 4A). As controls, three additional mutations for the outermost strands of blades I, II and III were engineered by replacing with the corresponding sequence of MMP-2 (MMP-9/IS4, MMP-9/IIS4 and MMP-9/IIIS4) (FIG. 4A). Employing a co-immunoprecipitation assay for COS-1 cells co-transfected with different combination of cDNAs, the inventors observed that mutations of blades I, II and III of MMP-9 PEX domain had no effect on MMP-9 homodimer formation; whereas mutation of blade IV (MMP-9/IVS4) failed to dimerize (FIG. 4B). This defect was not due to the loss of secretory ability of the mutant as evidenced by western blot and gelatin zymography of the conditioned medium from transfected COS-1 cells (FIG. 4B). The four mutants and wild type MMP-9 were then evaluated for their ability to enhance cell migration using a transwell migration assay (FIG. 4C). Mutation of the fourth β-strand of the PEX domain of MMP-9 (MMP-9/IVS4) failed to enhance COS-1 cells migration (FIG. 4C), as compared to wild type MMP-9, whereas mutations of blade II and III induced cell migration as well as wild type MMP-9. Unexpectedly, mutation of the first blade of the PEX domain of MMP-9 (MMP-9/1S4) also failed to induce cell migration. These data suggest that both blades I and IV of the MMP-9 PEX domain are required for the enhanced cell migration. Since the outermost β-strand of blade I is not involved in homodimer formation, another explanation for failure of migration of the MMP-9/IS4 mutant needs to be sought.

### Example 6

### Blocking MMP-9-Induced Cell Migration by Targeting the Homodimerization of MMP-9

Increased cell migration is involved in many pathological conditions including atherosclerosis and cancer; upregulated MMP-9 has been implicated in these processes (Stemlicht and Werb, 2001). One approach to blunt MMP-9 induced cell migration might be to target the herein discovered homodimer formation, which is a prerequisite for MMP-9-induced cell migration. Given the observation that the outermost β-strand of the fourth blade of the MMP-9 PEX domain is required for MMP-9 homodimerization formation, an 8-amino acids peptide (NQVDQVGY) (SEQ ID NO: 1) was synthesized to mimic the outer β-strand of blade IV of MMP-9 PEX domain (IVS4 peptide). As a control, a scrambled peptide with the same amino acids but rearranged in different order (VQYDNGQV) (SEQ ID NO: 51) was designed and synthesized. To test if IVS4 peptide blocked MMP-9 homodimer formation, COS-1 cells transfected with MMP-9 cDNA were treated with peptides for 30 minutes followed by a co-immunoprecipitation assay. As shown in FIG. 4D, the IVS4 peptide, but not scrambled peptide, efficiently blocked MMP-9 homodimer formation. To examine the potential therapeutic effect of this peptide, a transwell migration assay was performed. Interference with homodimerization by the peptide inhibited MMP-9-induced cell migration in a dose-dependent manner, whereas, the scrambled peptide had no apparent effect (FIGS. 4D & 4E). This IVS4 peptide, however, had no effect on cell migration induced by MT1-MMP, an integral membrane matrix metalloproteinase capable of stimulating cell migration (Cao et al., 2004). These data suggest that the IVS4 peptide specifically inhibits MMP-9-induced cell migration through interference with MMP-9 homodimer formation.

Results:

Increased cell migration is involved in many pathological conditions including atherosclerosis and cancer; upregulated MMP-9 has been implicated in these processes (Stemlicht and Werb, 2001). One approach to blunt MMP-9 induced cell migration might be to target homodimer formation, which is a prerequisite for MMP-9-induced cell migration. Given the observation that the outermost β-strand of the fourth blade of the MMP-9 PEX domain is required for MMP-9 homodimerization formation, an 8-amino acids peptide (NQVDQVGY) (SEQ ID NO: 1) was synthesized to mimic the outer β-strand of blade IV of MMP-9 PEX domain (IVS4 peptide). As a control, a scrambled peptide with the same amino acids but rearranged in different order (VQYDNGQV) (SEQ ID NO: 51) was designed and synthesized. To test if IVS4 peptide blocked MMP-9 homodimer formation, COS-1 cells transfected with MMP-9 cDNA were treated with peptides for 30 minutes followed by a co-immunoprecipitation assay. As shown in FIG. 4D, the IVS4 peptide, but not scrambled peptide, efficiently blocked MMP-9 homodimer formation. To examine the potential therapeutic effect of this peptide, a transwell migration assay was performed. Interference with homodimerization by the peptide inhibited MMP-9-induced cell migration in a dose-dependent manner, whereas, the scrambled peptide had no apparent effect (FIGS. 4D & 4E). This IVS4 peptide, however, had no effect on cell migration induced by MT1-MMP, an integral membrane matrix metalloproteinase capable of stimulating cell migration (Cao et al., 2004). These data suggest that the IVS4 peptide specifically inhibits MMP-9-induced cell migration through interference with MMP-9 homodimer formation.

### Example 7

### Cross-Talk Between MMP-9 and CD44 Regulates Cell Migration

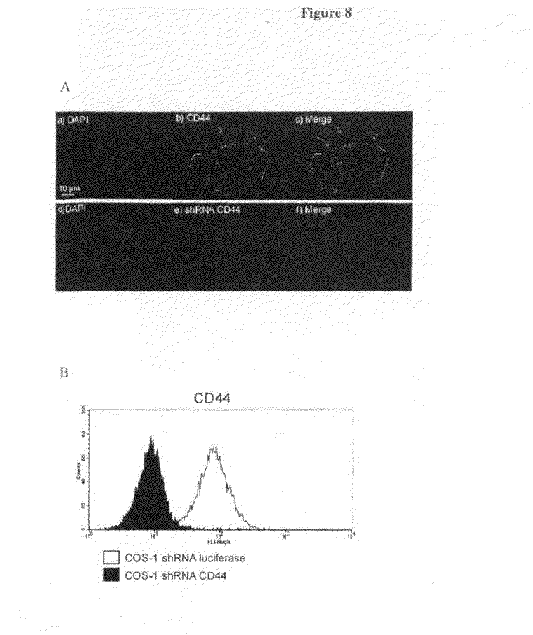

Although the outermost β-strand of blade I of MMP-9 is not required for MMP-9 homodimer formation (FIG. 4B), this motif is essential for MMP-9-induced cell migration (FIG. 4C). Given the evidence that MMP-9 interacts with CD44, a cell surface glycoprotein involved in cell-cell interactions, cell adhesion and migration (Yu and Stamenkovic, 1999, 2000; Redondo-Munoz et al., 2008), the inventors reasoned that MMP-9 may also form a heterodimer with CD44 which signals for cell migration. As examined by real time RT-PCR and immunofluorescent staining (FIG. 5B and FIG. 8A), COS-1 cells express endogenous CD44. Co-immunoprecipitation between CD44 and MMP-9 was assessed to determine the interaction between MMP-9 and CD44 in MMP-9 transfected COS-1 cells. Indeed, MMP-9 and CD44 co-precipitated in the transfected COS-1 cells (FIG. 5A), which confirms heterodimer formation of the two molecules. In contrast, swapping the PEX domain of MMP-9 by that of MMP-2 (MMP9/PEX_{MMP-2}/Myc) resulted in failure to complex with CD44, suggesting that heterodimer formation between CD44 and MMP-9 is through the PEX domain of MMP-9. To further determine which motif is required for the heterodimer formation, CD44 was co-expressed with MMP-9 mutants (MMP-9/IS4, MMP-9/IIS4, MMP-9/IIIS4 and MMP-9/IVS4) in transfected COS-1 cells, followed by co-immunoprecipitation. MMP-911S4 failed to form a complex with CD44, indicating that the outer β-strand of blade I interacts with CD44 at the cell surface (FIG. 5A). Mutations of outer β-strand of blades II, III and IV of the MMP-9 PEX domain did not interfere with CD44/MMP-9 heterodimerization.

To explore the role of CD44 in MMP-9-induced cell migration, the inventors silenced endogenous CD44 expression in COS-1 cells using a short hairpin RNA (shRNA) approach. As determined by a real time RT-PCR, mRNA of CD44 was suppressed more than 20 fold in CD44 shRNA expressing COS-1 cells (FIG. 5B). No detectable CD44 protein was found in COS-1 cells expressing CD44 shRNA examined by immunofluorescent staining and flow cytometry analysis (FIGS. 8A & 8B). By functional assay, the enhanced cell migration of MMP 9 transfected COS-1 cells was reduced significantly when CD44 was silenced, indicating that CD44 is a critical molecule in the MMP-9 cell migration signaling pathway (FIG. 5C). Interestingly, CD44 silenced COS-1 cells migrated to a relative higher levels as compared to shRNA luciferase control and wild type COS-1 cells. This observation might be due to decreased cell-cell and cell-matrix interactions by silence of CD44 expression in COS-1 cells (Acharya et al., 2008).

To further examine the importance of cross-talk between CD44 and MMP-9 in cell migration, an 8-amino acids peptide (SRPQGPFL) (SEQ ID NO: 2) was synthesized to mimic the outermost β-strand of the first blade of the MMP-9 PEX domain (IS4 peptide). As a control, a scrambled peptide with the same amino acids but rearranged in different order (GLSQPRFP) (SEQ ID NO: 35) was synthesized. COS-1 cells transfected with CD44 and MMP-9 cDNAs were treated with IS4 and IS4 scrambled peptides for 30 minutes followed by monitoring complex formations between CD44 and MMP-9 using a co-immunoprecipitation assay. IS4 peptide interfered with CD44/MMP-9 heterodimer, whereas the IS4 scrambled peptide had minimal effect (FIG. 5D). In addition, the IS4 peptide displayed dose-dependent inhibition of MMP-9-induced cell migration (FIG. 5E), but not the scrambled peptide (FIG. 5E). The potency of IS4 and IVS4 is IC₅₀: 12 μM and 50 μM, respectively. These mimicking inhibitory peptides demonstrate success in specifically targeting MMP-9, an important molecule in human disease.

Results:

Although the outermost β-strand of blade I of MMP-9 is not required for MMP-9 homodimer formation (FIG. 4B), this motif is essential for MMP-9-induced cell migration (FIG. 4C). Given the evidence that MMP-9 interacts with CD44, a cell surface glycoprotein involved in cell-cell interactions, cell adhesion and migration (Yu and Stamenkovic, 1999, 2000; Redondo-Munoz et al., 2008), the inventors reasoned that MMP-9 may also form a heterodimer with CD44 which signals for cell migration. As examined by real time RT-PCR and immunofluorescent staining (FIG. 5B and FIG. 8A), COS-1 cells express endogenous CD44. Co-immunoprecipitation between CD44 and MMP-9 was assessed to determine the interaction between MMP-9 and CD44 in MMP-9 transfected COS-1 cells. Indeed, MMP-9 and CD44 co-precipitated in the transfected COS-1 cells (FIG. 5A), which confirms heterodimer formation of the two molecules. In contrast, swapping the PEX domain of MMP-9 by that of MMP-2 (MMP9/PEX_{MMP-2}/Myc) resulted in failure to complex with CD44, suggesting that heterodimer formation between CD44 and MMP-9 is through the PEX domain of MMP-9. To further determine which motif is required for the heterodimer formation, CD44 was co-expressed with MMP-9 mutants (MMP-9/IS4, MMP-9/IIS4, MMP-9/IVS4 and MMP-9/IVS4) in transfected COS-1 cells, followed by co-immunoprecipitation. MMP-9/IS4 failed to form a complex with CD44, indicating that the outer β-strand of blade I interacts with CD44 at the cell surface (FIG. 5A). Mutations of outer β-strand of blades II, III and IV of the MMP-9 PEX domain did not interfere with CD44/MMP-9 heterodimerization.

To explore the role of CD44 in MMP-9-induced cell migration, the inventors silenced endogenous CD44 expression in COS-1 cells using a short hairpin RNA (shRNA) approach. As determined by a real time RT-PCR, mRNA of CD44 was suppressed more than 20 fold in CD44 shRNA expressing COS-1 cells (FIG. 5B). No detectable CD44 protein was found in COS-1 cells expressing CD44 shRNA examined by immunofluorescent staining and flow cytometry analysis (FIGS. 8A & 8B). By functional assay, the enhanced cell migration of MMP-9 transfected COS-1 cells was reduced significantly when CD44 was silenced, indicating that CD44 is a critical molecule in the MMP-9 cell migration signaling pathway (FIG. 5C). Interestingly, CD44 silenced COS-1 cells migrated to a relative higher levels as compared to shRNA luciferase control and wild type COS-1 cells. This observation might be due to decreased cell-cell and cell-matrix interactions by silence of CD44 expression in COS-1 cells (Acharya et al., 2008).

To further examine the importance of cross-talk between CD44 and MMP-9 in cell migration, an 8-amino acids peptide (SRPQGPFL) (SEQ ID NO: 2) was synthesized to mimic the outermost β-strand of the first blade of the MMP-9 PEX domain. As a control, a scrambled peptide with the same amino acids but rearranged in different order (GLSQPRFP) (SEQ ID NO: 35) was synthesized. COS-1 cells transfected with CD44 and MMP-9 cDNAs were treated with IS4 and IS4 scrambled peptides for 30 minutes followed by monitoring complex formations between CD44 and MMP-9 using a co-immunoprecipitation assay. IS4 peptide interfered with CD44/MMP-9 heterodimer, whereas the IS4 scrambled peptide had minimal effect (FIG. 5D). In addition, the IS4 peptide displayed dose-dependent inhibition of MMP-9-induced cell migration (FIG. 5E), but not the scrambled peptide (FIG. 5E). Although the potency of IS4 and IVS4 peptides (IC₅₀: 12 μM and 50 μM, respectively) are suboptimal from a therapeutic standpoint, generation of the mimicking inhibitory peptides provide proof of concept for specific targeting an important molecule in human disease.

### Example 8

### MMP-9-CD44-EGFR Axis in Cell Migration

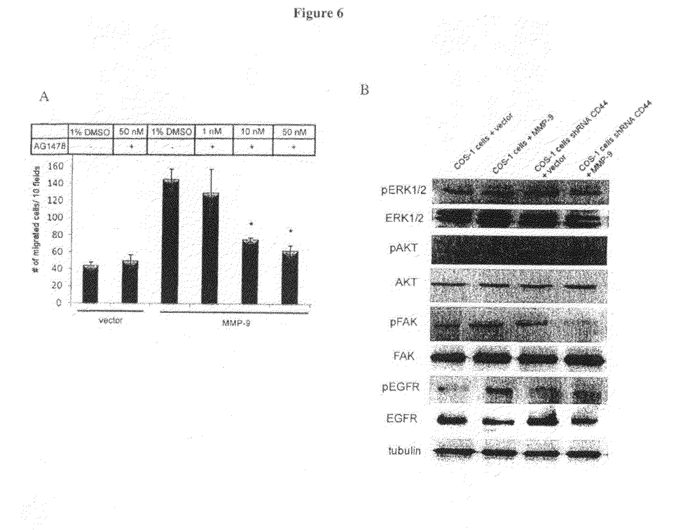

It has been reported that CD44 activates receptor tyrosine kinases (RTKs) initiating signaling for cell migration (Kim et al., 2008). To further investigate which RTKs are involved in the MMP-9-CD44 signaling pathway for cell migration, small molecule inhibitors targeting different RTKs were employed and tested in a transwell migration assay. Among these tested inhibitors, an EGFR inhibitor (AG1478) significantly inhibited cell migration in MMP-9 transfected COS-1 cells in a dose-dependent manner as compared to the vector control (FIG. 6A). This data suggests that EGFR may be activated in MMP-9 transfected COS-1 cells. To test if MMP-9-CD44 interactions results in the activation of EGFR, COS-1 and CD44-silenced COS-1 cells transfected with MMP-9 as well as vector control were examined by western blotting using a specific anti-phosphorylated EGFR antibody. As shown in FIG. 6B, expression of MMP-9 in wild type COS-1 cells resulted in increased phosphorylation of EGFR as compared to vector control. This activation of EGFR did not occurred in CD44-silenced COS-1 cells transfected with MMP-9 cDNA. These data are consistent with a pathway by which complex formation between MMP-9 and CD44 leads to activation of EGFR.

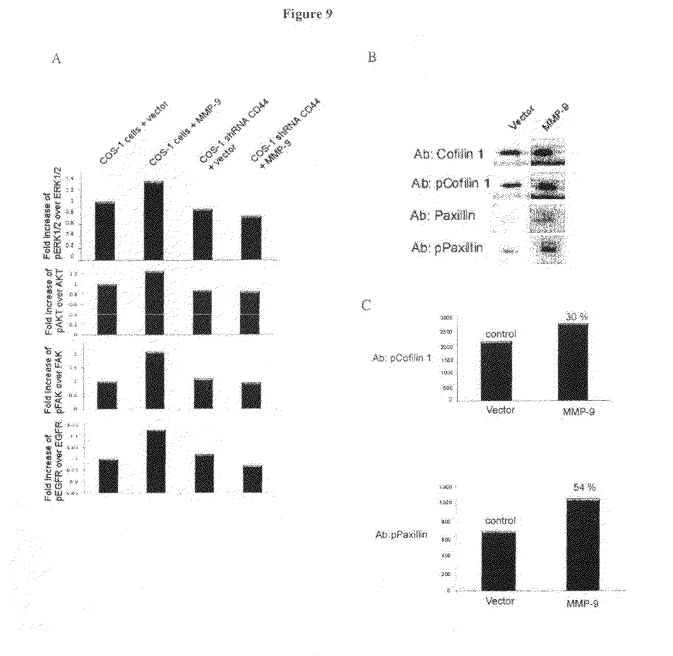

To further dissect the MMP-9-CD44-EGFR pathway, the inventors examined downstream effectors of activated EGFR, including pERK, pAKT, and pFAK, which have been implicated in EGFR-induced cell migration (Kim et al., 2008). An increase of pERK1/2, pAKT, pFAK and pEGFR was observed in MMP-9 transfected COS-1 cells, but not MMP-9 transfected CD44-silenced COS-1 cells or vector control, confirming MMP-9-CD44-EGFR signaling axis in MMP-9-induced cell migration (FIG. 6B & FIG. 9A).

In another approach to identify signaling pathway(s) differentially activated in MMP-9 expressing COS-1 cells, the inventors employed Kinexus Antibody Microarrays. These arrays simultaneously detected the presence and relative quantities of 500 pan-specific and 300 phospho-site antibodies in MMP-9 transfected COS-1 cells, as compared to vector control cells. The inventors confirmed elevated activity of EGFR, pFAK, pERK, pAKT in MMP-9 transfected COS-1 cells (FIG. 9A). In addition, a series of migration related proteins were also increased, including pCofilin 1 and pPaxillin (30% and 54% increase, respectively) (FIGS. 9B & 9C).

Taken together, this study demonstrates that MMP-9-enhanced protease-independent cell migration involves the coordination of PEX domain homodimerization and CD44 heterodimerization leading to EGFR activation.

Results:

It has been reported that CD44 activates receptor tyrosine kinases (RTKs) initiating signaling for cell migration (Kim et al., 2008). To further investigate which RTKs are involved in the MMP-9-CD44 signaling pathway for cell migration, small molecule inhibitors targeting different RTKs were employed and tested in a transwell migration assay. Among these tested inhibitors, an EGFR inhibitor (AG1478) significantly inhibited cell migration in MMP-9 transfected COS-1 cells in a dose-dependent manner as compared to the vector control (FIG. 6A). This data suggests that EGFR may be activated in MMP-9 transfected COS-1 cells. To test if MMP-9-CD44 interactions results in the activation of EGFR, COS-1 and CD44-silenced COS-1 cells transfected with MMP-9 as well as vector control were examined by western blotting using a specific anti-phosphorylated EGFR antibody. As shown in FIG. 6B, expression of MMP-9 in wild type COS-1 cells resulted in increased phosphorylation of EGFR as compared to vector control. This activation of EGFR did not occurred in CD44-silenced COS-1 cells transfected with MMP-9 cDNA. These data are consistent with a pathway by which complex formation between MMP-9 and CD44 leads to activation of EGFR.

To further dissect the MMP-9-CD44-EGFR pathway, the inventors examined downstream effectors of activated EGFR, including pERK, pAKT, and pFAK, which have been implicated in EGFR-induced cell migration (Kim et al., 2008). An increase of pERK1/2, pAKT, pFAK and pEGFR was observed in MMP-9 transfected COS-1 cells, but not MMP-9 transfected CD44-silenced COS-1 cells or vector control, confirming MMP-9-CD44-EGFR signaling axis in MMP-9-induced cell migration (FIG. 6B & FIG. 9A).

In another approach to identify signaling pathway(s) differentially activated in MMP-9 expressing COS-1 cells, the inventors employed Kinexus Antibody Microarrays. These arrays simultaneously detected the presence and relative quantities of 500 pan-specific and 300 phospho-site antibodies in MMP-9 transfected COS-1 cells, as compared to vector control cells. The inventors confirmed elevated activity of EGFR, pFAK, pERK, pAKT in MMP-9 transfected COS-1 cells (FIG. 9A). In addition, a series of migration related proteins were also increased, including pCofilin 1 and pPaxillin (30% and 54% increase, respectively) (FIGS. 9B & 9C).

### Example 9

Inhibition of HT1080 Cell Migration by Synthetic Peptides Interfering with MMP-9 Dimerizations.

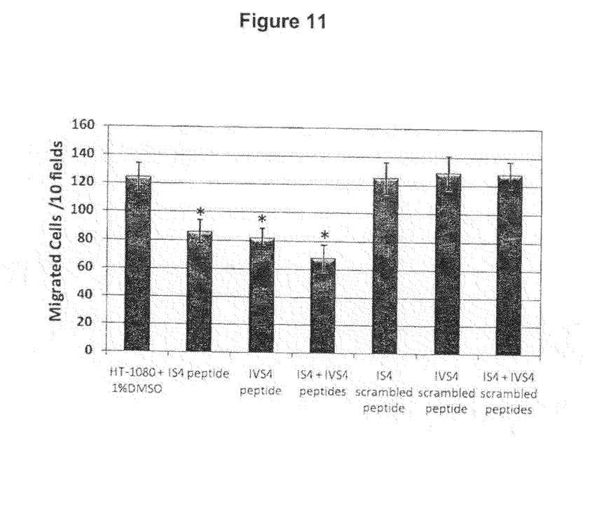

Human fibrosarcoma HT1080 cells are highly invasive and metastatic. Various proteases, including urokinase plasminogen activator (uPA), MMP-2, -9, and -14, have been found to play important roles in HT1080 cell invasion. Since this cell line produces endogenous MMP-9, the inventors sought to test if the MMP-9 IS4 and IVS4 peptides can be used to interfere with function of endogenous MMP-9. To this end, HT1080 cells were employed and cell migratory ability was evaluated using a Transwell chamber migration assay (FIG. 11). The cells were incubated with 100 μM of IS4 peptide (SRPQGPFL) (SEQ ID NO: 2), IS4 scrambled peptide (GLSQPRFP) (SEQ ID NO: 35), IVS4 peptide (NQVDQVGY) (SEQ ID NO: 1), IVS4 scrambled peptide (VQYDNGQV) (SEQ ID NO: 51), and a combination of IS4 and IVS4 peptides for 6 h at 37° C. 1% DMSO was used as a vehicle control. Migrated cells were microscopically counted. Each data point was performed in triplicate and the experiment was repeated three times (*P<0.05). FIG. 11 shows that the IS4 and IVS4 peptides of MMP9 significantly reduced HT1080 cell migration. A combination of IS4 and IVS4 did not further reduce HT1080 cell migration. While not limiting the invention to any particular mechanism, it is the inventors' view that this is potentially because both inhibitory peptides target the same molecule.

Taken together, the data demonstrate that MMP-9-enhanced protease-independent cell migration involves the coordination of PEX domain homodimerization and CD44 heterodimerization leading to EGFR activation.

### REFERENCES

Acharya, P. S., Majumdar, S., Jacob, M., Hayden, J., Mrass, P., Weninger, W., Assoian, R. K., and Pure, E. (2008). Fibroblast migration is mediated by CD44-dependent TGF beta activation. J Cell Sci 121, 1393-1402.
Bjorklund, M., Heikkila, P., and Koivunen, E. (2004). Peptide inhibition of catalytic and noncatalytic activities of matrix metalloproteinase-9 blocks tumor cell migration and invasion. J Biol Chem 279, 29589-29597.
Bjorklund, M., and Koivunen, E. (2005). Gelatinase-mediated migration and invasion of cancer cells. Biochim Biophys Acta 1755, 37-69.
Bourguignon, L. Y., Gilad, E., Brightman, A., Diedrich, F., and Singleton, P. (2006). Hyaluronan-CD44 interaction with leukemia-associated RhoGEF and epidermal growth factor receptor promotes Rho/Ras co-activation, phospholipase C epsilon-Ca2+ signaling, and cytoskeleton modification in head and neck squamous cell carcinoma cells. J Biol Chem 281, 14026-14040.
Cao, J., Chiarelli, C., Richman, 0., Zarrabi, K., Kozarekar, P., and Zucker, S. (2008). Membrane type 1 matrix metalloproteinase induces epithelial-to-mesenchymal transition in prostate cancer. J. Biol. Chem. 283, 6232-6240.
Cao, J., Drews, M., Lee, H. M., Conner, C., Bahou, W. F., and Zucker, S. (1998). The propeptide domain of membrane type 1 matrix metalloproteinase is required for binding of tissue inhibitor of metalloproteinases and for activation of pro-gelatinase A. J. Biol. Chem. 273, 34745-34752.
Cao, J., Kozarekar, P., Pavlaki, M., Chiarelli, C., Bahou, W. F., and Zucker, S. (2004). Distinct roles for the catalytic and hemopexin domains of membrane type 1-matrix metalloproteinase in substrate degradation and cell migration. J. Biol. Chem. 279, 14129-14139.
Cao, J., Rehemtulla, A., Bahou, W., and Zucker, S. (1996). Membrane type matrix metalloproteinase 1 activates pro-gelatinase A without furin cleavage of the N-terminal domain J. Biol. Chem. 271, 30174-30180.
Cha, H., Kopetzki, E., Huber, R., Lanzendorfer, M., and Brandstetter, H. (2002). Structural basis of the adaptive molecular recognition by MMP9. J Mol Biol 320, 1065-1079.
Collier, I. E., Saffarian, S., Manner, B. L., Elson, E. L., and Goldberg, G. (2001). Substrate recognition by gelatinase A: the C-terminal domain facilitates surface diffusion. Biophys J 81, 2370-2377.
Coussens, L. M., Fingleton, B., and Matrisian, L. M. (2002). Matrix metalloproteinase inhibitors and cancer: trials and tribulations. Science 295, 2387-2392.
Davis, B. G. (2004). Biochemistry Mimicking posttranslational modifications of proteins. Science 303, 480-482.
Dufour, A., Sampson, N. S., Zucker, S., and Cao, J. (2008). Role of the hemopexin domain of matrix metalloproteinases in cell migration. J. Cell Physiol 217, 643-651.
Ellerbroek, S. M., Halbleib, J. M., Benavidez, M., Warmka, J. K., Wattenberg, E. V., Stack, M. S., and Hudson, L. G. (2001). Phosphatidylinositol 3-kinase activity in epidermal growth factor-stimulated matrix metalloproteinase-9 production and cell surface association. Cancer Res 61, 1855-1861.
Ezhilarasan, R., Jadhav, U., Mohanam, I., Rao, J. S., Gujrati, M., and Mohanam, S. (2009). The hemopexin domain of WIMP-9 inhibits angiogenesis and retards the growth of intracranial glioblastoma xenograft in nude mice. Int J Cancer 124, 306-315.
Goldberg, G. I., Strongin, A., Collier, I. E., Genrich, L. T., and Manner, B. L. (1992). Interaction of 92-kDa type IV collagenase with the tissue inhibitor of metalloproteinases prevents dimerization, complex formation with interstitial collagenase, and activation of the proenzyme with stromelysin. J Biol Chem 267, 4583-4591.
Kim, Y., Lee, Y. S., Choe, J., Lee, H., Kim, Y. M., and Jeoung, D. (2008). CD44-epidermal growth factor receptor interaction mediates hyaluronic acid-promoted cell motility by activating protein kinase C signaling involving Akt, Racl, Phox, reactive oxygen species, focal adhesion kinase, and MMP-2. J Biol Chem 283, 22513-22528.
Mantuano, E., Inoue, G., Li, X., Takahashi, K., Gaultier, A., Gonias, S. L., and Campana, W. M. (2008). The hemopexin domain of matrix metalloproteinase-9 activates cell signaling and promotes migration of schwann cells by binding to low-density lipoprotein receptor-related protein. J Neurosci 28, 11571-11582.
Marcoux, N., and Vuori, K. (2005). EGF receptor activity is essential for adhesion-induced stress fiber formation and cofilin phosphorylation. Cell Signal 17, 1449-1455.
Massova, I., Kotra, L. P., Fridman, R., and Mobashery, S. (1998). Matrix metalloproteinases: structures, evolution, and diversification. FASEB J 12, 1075-1095.
Monferran, S., Paupert, J., Dauvillier, S., Salles, B., and Muller, C. (2004). The membrane form of the DNA repair protein Ku interacts at the cell surface with metalloproteinase 9. EMBO J. 23, 3758-3768.
Morgunova, E., Tuuttila, A., Bergmann, U., and Tiyggvason, K. (2002). Structural insight into the complex formation of latent matrix metalloproteinase 2 with tissue inhibitor of metalloproteinase 2. Proc Natl Acad Sci USA 99, 7414-7419.
Morrison, C. J., Butler, G. S., Rodriguez, D., and Overall, C. M. (2009). Matrix metalloproteinase proteomics: substrates, targets, and therapy. Curr Opin Cell Biol 21, 645-653.
Murai, T., Miyauchi, T., Yanagida, T., and Sako, Y. (2006). Epidermal growth factor-regulated activation of Rac GTPase enhances CD44 cleavage by metalloproteinase disintegrin ADAM10. Biochem J 395, 65-71.
Nagase, H., and Woessner, J. F., Jr. (1999). Matrix metalloproteinases. J Biol Chem 274, 21491-21494.
Olson, M. W., Bernardo, M. M., Pietila, M., Gervasi, D. C., Toth, M., Kotra, L. P., Massova, I., Mobashery, S., and Fridman, R. (2000). Characterization of the monomeric and dimeric forms of latent and active matrix metalloproteinase-9. Differential rates for activation by stromelysin 1. J Biol Chem 275, 2661-2668.
Olson, M. W., Toth, M., Gervasi, D. C., Sado, Y., Ninomiya, Y., and Fridman, R. (1998). High affinity binding of latent matrix metalloproteinase-9 to the alpha2(IV) chain of collagen IV. J Biol Chem 273, 10672-10681.
Pavlaki, M., and Zucker, S. (2003). Matrix metalloproteinase inhibitors (MMPIs): the beginning of phase I or the termination of phase III clinical trials. Cancer Metastasis Rev 22, 177-203.
Redondo-Munoz, J., Ugarte-Berzal, E., Garcia-Marco, J. A., del Cerro, M. H., Van den Steen, P. E., Opdenakker, G., Terol, M. J., and Garcia-Pardo, A. (2008). Alpha4beta1 integrin and 190-kDa CD44v constitute a cell surface docking complex for gelatinase B/MMP-9 in chronic leukemic but not in normal B cells. Blood 112, 169-178.
Roderfeld, M., Graf, J., Giese, B., Salguero-Palacios, R., Tschuschner, A., Muller-Newen, G., and Roeb, E. (2007). Latent MMP-9 is bound to TIMP-1 before secretion. Biol Chem 388, 1227-1234.
Samanna, V., Ma, T., Mak, T. W., Rogers, M., and Chellaiah, M. A. (2007). Actin polymerization modulates CD44 surface expression, MMP-9 activation, and osteoclast function. J Cell Physiol 213, 710-720.
Stefanidakis, M., Karjalainen, K., Jaalouk, D. E., Gahmberg, C. G., O'Brien, S., Pasqualini, R., Arap, W., and Koivunen, E. (2009). Role of leukemia cell invadosome in extramedullary infiltration. Blood 114, 3008-3017.
Sternlicht, M. D., and Werb, Z. (2001). How matrix metalloproteinases regulate cell behavior. Annu Rev Cell Dev Biol 17, 463-516.
Tsukita, S., Oishi, K., Sato, N., Sagara, J., and Kawai, A. (1994). ERM family members as molecular linkers between the cell surface glycoprotein CD44 and actin-based cytoskeletons. J Cell Biol 126, 391-401.
Van den Steen, P. E., Van Aelst, I., Hvidberg, V., Piccard, H., Fiten, P., Jacobsen, C., Moestrup, S. K., Fry, S., Royle, L., Wormald, M. R., Wallis, R., Rudd, P. M., Dwek, R. A., and Opdenakker, G. (2006). The hemopexin and O-glycosylated domains tune gelatinase B/MMP-9 bioavailability via inhibition and binding to cargo receptors. J Biol Chem 281, 18626-18637.
Yao, J. S., Chen, Y., Zhai, W., Xu, K., Young, W. L., and Yang, G. Y. (2004). Minocycline exerts multiple inhibitory effects on vascular endothelial growth factor-induced smooth muscle cell migration: the role of ERK1/2, PI3K, and matrix metalloproteinases. Circ Res 95, 364-371.
Yu, Q., and Stamenkovic, I. (1999). Localization of matrix metalloproteinase 9 to the cell surface provides a mechanism for CD44-mediated tumor invasion. Genes Dev 13, 35-48.
Yu, Q., and Stamenkovic, I. (2000). Cell surface-localized matrix metalloproteinase-9 proteolytically activates TGF-beta and promotes tumor invasion and angiogenesis. Genes Dev 14, 163-176.
Zucker, S., Cao, J., and Chen, W. T. (2000). Critical appraisal of the use of matrix metalloproteinase inhibitors in cancer treatment. Oncogene 19, 6642-6650.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiment, it should be understood that the invention as claimed should not be unduly limited to such specific embodiments. Indeed, various modifications of the described modes for carrying out the invention which are obvious to those skilled in the art and in fields related thereto are intended to be within the scope of the following claims.

We claim:

1. A composition comprising a polypeptide that
1) comprises a sequence selected from the group consisting of at least a portion of NQVDQVGY (SEQ ID NO:1) and at least a portion of SRPQGPFL (SEQ ID NO:2), and
2) lacks at least a portion of MMP-9 hemopexin domain sequence, wherein said portion of MMP-9 hemopexin domain is selected from the group consisting of
a) at least a portion of
(SEQ ID NO: 4)
DACNVNIFDAIAEIGNQLYLFKDGKYWRFSEGRG,
b) at least a portion of
(SEQ ID NO: 5)
IADKWPALPRKLDSVFEERLSKKLFFFSGRQVWVYTGASVLGPRRLDK
LGLGADVAQVTGALRSGRGKMLLFSGRRLWRFDVKAQMVDPRSASE
VDRMFPGVPLDTHDVFQYREKAYFCQDRFYWRVSSRSEL,
and
c) at least a portion of VTYDILQCPED (SEQ ID NO:6).

2. The composition of claim 1, wherein said portion of NQVDQVGY (SEQ ID NO:1) is selected from the group consisting of NQVDQVG (SEQ ID NO: 7), NQVDQV (SEQ ID NO: 8), NQVDQ (SEQ ID NO: 9), NQVD (SEQ ID NO: 10), QVDQVGY (SEQ ID NO: 11), VDQVGY (SEQ ID NO: 12), DQVGY (SEQ ID NO: 13), QVGY (SEQ ID NO: 14), QVDQVG (SEQ ID NO: 15), VDQVG (SEQ ID NO: 16) DQVG (SEQ ID NO: 17), QVDQV (SEQ ID NO: 18), VDQV (SEQ ID NO: 19), and QVDQ (SEQ ID NO: 20).

3. The composition of claim 1, wherein said portion of SRPQGPFL (SEQ ID NO:2) is selected from the group consisting of SRPQGPF (SEQ ID NO: 21), SRPQGP (SEQ ID NO: 22), SRPQG (SEQ ID NO: 23), SRPQ (SEQ ID NO: 24), RPQGPFL (SEQ ID NO: 25), PQGPFL (SEQ ID NO: 26), QGPFL (SEQ ID NO: 27), GPFL (SEQ ID NO: 28), RPQGPF (SEQ ID NO: 29), PQGPF (SEQ ID NO: 30), QGPF (SEQ ID NO: 31), RPQGP (SEQ ID NO: 32), PQGP (SEQ ID NO: 33), and RPQG (SEQ ID NO: 34).

4. A composition comprising a polypeptide that consists of a sequence selected from the group consisting of at least a portion of NQVDQVGY (SEQ ID NO:1) and at least a portion of SRPQGPFL (SEQ ID NO:2).

5. The composition of claim 4, wherein said polypeptide has been modified to resist proteolysis.

6. The composition of claim 5, where said polypeptide has been terminally modified.

7. A method for reducing one or more symptoms of disease in a subject, comprising
a) providing
i) a mammalian subject in need of reducing one or more symptoms of disease, and
ii) a composition selected from the group consisting of the composition of claim 1 and the composition of claim 4, and
b) administering to said subject a therapeutic amount of said composition to produce a treated subject, wherein said administering is under conditions for reducing one or more symptoms of said disease.

8. The method of claim 7, further comprising c) detecting a reduction in one or more symptoms of said disease in said treated subject.

9. The method of claim 7, wherein one or more symptoms of said disease comprise increased cell migration in the presence of MMP-9 compared to in the absence of MMP-9.

10. The method of claim 9, wherein said therapeutic amount of said composition specifically reduces said cell migration.

11. The method of claim 7, wherein said composition comprises an amount of said at least a portion of NQVDQVGY (SEQ ID NO:1) that reduces homodimerization of MMP-9.

12. The method of claim 7, wherein said composition comprises an amount of said at least a portion of SRPQGPFL (SEQ ID NO:2) that reduces heterodimerization of MMP-9 and CD44.

13. The method of claim 9, wherein said cell is a cancer cell.

14. The method of claim 13, wherein said cancer cell is a metastatic cancer cell.

15. A method for reducing cell migration, comprising
a) providing
i) a cell expressing MMP-9, and
ii) a composition selected from the group consisting of the composition of claim 1 and the composition of claim 4, and
b) administering said composition to said cell under conditions for reducing migration of said cell.

16. The method of claim 15, further comprising c) detecting reduced migration of said cell.

17. The method of claim 15, wherein migration of said cell is increased in the presence of MMP-9 compared to in the absence of MMP-9, and wherein said composition specifically reduces said cell migration.

18. The method of claim 17, wherein said composition comprises an amount of said at least a portion of NQVDQVGY (SEQ ID NO:1) that reduces homodimerization of MMP-9.

19. The method of claim 17, wherein said composition comprises an amount of said at least a portion of SRPQGPFL (SEQ ID NO:2) that reduces heterodimerization of MMP-9 and CD44.

20. A method for treating a cancer at risk of metastases in a subject, comprising
a) providing
i) a mammalian subject having cancer at risk of metastases, and
ii) a composition selected from the group consisting of the composition of claim 1 and the composition of claim 4, and
b) administering to said subject a therapeutic amount of said composition to produce a treated subject, wherein said administering is under conditions for reducing said risk of metastases.
